SEQUENCE LISTING (15 sequences)

SEQ ID NO: 1 - protein, 5 aa, Artificial Sequence
  Synthetic peptide
RFWMS

SEQ ID NO: 2 - protein, 17 aa, Artificial Sequence
  Synthetic peptide
EINPESSTINYTPSLKD

SEQ ID NO: 3 - protein, 10 aa, Artificial Sequence
  Synthetic peptide
SASSSVRYMH

SEQ ID NO: 4 - protein, 7 aa, Artificial Sequence
  Synthetic peptide
DTSEKAS

SEQ ID NO: 5 - protein, 9 aa, Artificial Sequence
  Synthetic peptide
QQWSSHPFT

SEQ ID NO: 6 - protein, 87 aa, Artificial Sequence
  Synthetic peptide
QIVLTQSPAIMSASPGEKVTMTCSASSSVRYMHWYQQKSGTSPKRWIYDTSEKASGVPTRFSGSGSGTSYSLTISTMEAEDAATYYC

SEQ ID NO: 7 - protein, 98 aa, Artificial Sequence
  Synthetic peptide
EVKLLESGGGLVQPGGSLKLSCAASGFDFSRFWMSWVRQAPGKGLEWIGEINPESSTINYTPSLKDKFIISRDNAKNTLYLQMSKVRSEDTALYYCAR

SEQ ID NO: 8 - DNA, 708 bp, Mus musculus
atggattttcaggtgcagattttcagcttcctgctaatcagtgcctcagtcataatatccagaggacaaattgttctcacccagtctccagcaatcatgtctgcatctccaggggagaaggtcaccatgacctgcagtgccagctcaagtgtacgttacatgcactggtaccagcagaagtcaggcacctcccccaaaagatggatttatgacacatccgaaaaggcttctggagtccctactcgcttcagtggcagtgggtctgggacctcttactctctcacaatcagcaccatggaggctgaagatgctgccacttattactgccagcagtggagtagtcacccattcacgttcggctcggggacaaaattggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttag

SEQ ID NO: 9 - DNA, 294 bp, Mus musculus
gaggtgaagcttctcgagtctggaggtggcctggtgcagcctggaggatccctgaaactctcctgtgcagcctcaggattcgattttagtagattctggatgagttgggtccggcaggctccagggaaagggctagaatggattggagaaattaatccagagagcagtacgataaactatacgccatctctaaaggataaattcataatctccagagacaacgccaaaaatacgctgtacctgcaaatgagcaaagtgagatctgaggacacagccctttattactgtgcaaga

SEQ ID NO: 10 - DNA, 23 bp, Artificial Sequence
  Primer
atggayttycargtncarathtt

SEQ ID NO: 11 - DNA, 21 bp, Artificial Sequence
  Primer
yttytgytgrtaccartgcat

SEQ ID NO: 12 - DNA, 22 bp, Artificial Sequence
  Primer
ctggtgcagcctggaggatccc

SEQ ID NO: 13 - DNA, 21 bp, Artificial Sequence
  Primer
tcttgcacagtaataaagggc

SEQ ID NO: 14 - protein, 11 aa, Artificial Sequence
  Synthetic peptide
DAGPYGNYFDY

SEQ ID NO: 15 - protein, 557 aa, Mus musculus
EVKLLESGGGLVQPGGSLKLSCAASGFDFSRFWMSWVRQAPGKGLEWIGEINPESSTINYTPSLKDKFIISRDNAKNTLYLQMSKVRSEDTALYYCARDAGPYGNYFDYWGQGTTLTVSSESQSFPNVFPLVSCESPLSDKNLVAMGCLARDFLPSTISFTWNYQNNTEVIQGIRTFPTLRTGGKYLATSQVLLSPKSILEGSDEYLVCKIHYGGKNRDLHVPIPAVAEMNPNVNVFVPPRDGFSGPAPRKSKLICEATNFTPKPITVSWLKDGKLVESGFTTDPVTIENKGSTPQTYKVISTLTISEIDWLNLNVYTCRVDHRGLTFLKNVSSTCAASPSTDILTFTIPPSFADIFLSKSANLTCLVSNLATYETLNISWASQSGEPLETKIKIMESHPNGTFSAKGVASVCVEDWNNRKEFVCTVTHRDLPSPQKKFISKPNEVHKHPPAVYLLPPAREQLNLRESATVTCLVKGFSPADISVQWLQRGQLLPQEKYVTSAPMPEPGAPGFYFTHSILTVTEEEWNSGETYTCVVGHEALPHLVTERTVDKSTGK

(19) United States
(12) Patent Application Publication
(10) Pub. No.: US 2012/0263719 A1
(43) Pub. Date: Oct. 18, 2012

(54) COMPOSITIONS AND METHODS FOR TREATING ASPERGILLOSIS

(75) Inventors: David Mirelman, Ramat Efal (IL); Aharon Rabinkov, Rehovot (IL); Elena Appel, Rehovot (IL)

(73) Assignee: YEDA RESEARCH AND DEVELOPMENT CO., LTD., Rehovot (IL)

(21) Appl. No.: 13/502,794

(22) PCT Filed: Oct. 21, 2010
(86) PCT No.: PCT/IL10/00872

§ 371 Date: Jul. 2, 2012

Related U.S. Application Data
(60) Provisional application No. 61/253,871, filed on Oct. 22, 2009.

(51) Int. Cl. A61K 39/395; C12N 9/96; C12N 9/88; C12N 15/13; A61P 11/00; C12N 5/16; C12N 1/21; C12N 1/19; C12N 5/10; A61P 31/10; C07K 16/14; C12N 15/63

(52) U.S. Cl. 424/134.1; 530/388.5; 530/387.3; 435/188; 435/232; 536/23.53; 435/320.1; 435/341; 435/252.3; 435/254.2; 435/414; 435/411; 424/152.1; 424/178.1

(57) ABSTRACT

The present invention provides monoclonal antibodies specific for members of the Aspergillus genus and uses thereof for increasing the survival rate of a subject infected with an Aspergillus species. The present invention also provides conjugates of alliinase with monoclonal antibodies specific for members of the Aspergillus genus that preserve the ability of the antibody to recognize the fungus as well as the activity of the enzyme to produce cytotoxic molecules of allicin from the substrate alliin. The invention further provides pharmaceutical compositions comprising the conjugates of the mAbs of the invention and alliinase and uses thereof in combination with the substrate alliin in the treatment of diseases cause by Aspergillus species, in particular invasive aspergillosis.

### FIELD OF THE INVENTION

The present invention relates to monoclonal antibodies against Aspergillus and to conjugates and fusion proteins comprising them, particularly conjugates and fusion proteins with alliinase, to pharmaceutical compositions comprising these antibodies and to their use in preventing and treating of diseases caused by Aspergillus species.

### BACKGROUND OF THE INVENTION

Aspergillus fumigatus (AF) is an opportunistic fungal pathogen that is responsible for invasive aspergillosis (IA) in immunocompromised individuals (Kedziora et al., 2008, Pneumonol. Alergol. Pol. 76:4000-6 and Latge et al., 2001, Trends in Microbiol., 9:382-9). Patients with hematological or solid malignancies, AIDS as well as bone marrow and organ transplant recipients are particularly vulnerable to the infection. Pulmonary infection by AF airborne conidia, which germinate and grow in the small airways and alveolar spaces, is the predominant cause of IA (Latge et al., 2001, Trends in Microbiol., 9:382-9). Studies of post mortem lung specimens have shown AF to be present and viable more frequently than would be expected from its prevalence among the fungal conidia found normally in the air (Muillins and Seaton, 1978, Clin. Allergy 8:525-33). In healthy individuals a number of shared defense mechanisms are activated in response to a range of fungi. Neutrophils, alveolar macrophages and monocyte effector cells have a fundamental antifungal function (Romani, 2008, Med. Mycol. 46:515-29). Resident and monocyte-derived macrophages ingest and kill Aspergillus conidia, thus preventing transition into the invasive hyphal form (Behnsen et al., 2007, PLoS Pathogens 3:e3; Segal, 2007, Oncologist, 12:7-13). Treatments with high doses of corticosteroids, which suppress neutrophil and macrophage function, predispose patients to invasive pulmonary aspergillosis (IPA) (Lionakis and Kontoyiannis, 2003, Lancet, 362:1828-38). In many such cases, bronchoalveolar macrophages fail to control the fungus (Wasylnka and Moore, 2003, J. Cell Sci., 116:1579-87), conidia germinate into hyphae, pierce the thin alveolar barrier and invade the underlying blood vessels.

Numerous antifungal strategies to treat IPA infections have been reported. Most are based on azoles (such as voriconazole), amphotericin (AMB) or echinocandins. Recent studies in animal models have demonstrated a therapeutic potential in several fungal infections, for monoclonal antibodies (Torosantucci et al., 2009, PLoS One 4:e5392) as well as for ultrashort synthetic lipopeptides (Vallon-Eberhard et al., 2008, Antimicrob. Agents Chemother., 52:3118-26). Anti-idiotypic antibodies where found to effectively control AF infection in neutropenic mice when administered intranasally (Cenci et al., 2002, Infect. Immun. 70:2375-82). Furthermore, a combination of an antifungal HSP90 antibody with AMB improved the recovery in patients with invasive Candidiasis (Matthews et al., 2003, Antimicrob. Agents Chemother., 47:2208-16).

Currently, despite advances in early diagnosis and new antifungal agents, IA remains a leading cause of death in the immunocompromised patient population, with an attributed mortality rate ranging from 30% to 80% (Enoch et al., 2006, J Med. Microbiol. 55:809-18)

Allicin, (diallyl-dithiosulfinate) the biologically active molecule of garlic has been shown to have a very wide range of antimicrobial activities and contributes to the defense of the garlic plant from soil microorganisms (Ankri and Mirelman, 1999, Microbes Infect., 1:125-9). Allicin is produced by the catalytic reaction of the enzyme alliinase (E.C.4.4.1.4), with the inert, non-protein amino acid substrate, alliin [(+)S-Allyl-cysteine sulfoxide]. Crushing of a garlic clove breaks down the compartmentalization and brings the enzyme and its substrate into contact, leading to allicin production (Miron et al., 2006, Anal. Biochem. 351:152-4). The potential of allicin as an anti-Aspergillus agent in vivo was shown (Shadkchan et al., 2004, Antimicrob. Agents Chemother., 53:832-6). Despite its short half-life, five repetitive i.v. administrations of allicin significantly prolonged the survival of mice infected with AF. The delivery of allicin, however, remains a major concern due to its instability in blood circulation. Allicin rapidly transforms into secondary products that lack the antimicrobial activity following intravenous injection (Freeman and Kodera, 1995, J. Agric. Food Chem. 43:2332-8).

Some inventors of the present invention have disclosed the use of conjugates of alliinase with a protein carrier that targets the alliinase to specific cells (U.S. Pat. No. 7,445,802). Specifically, it was shown that a monoclonal antibody which recognizes the ErbB-2 receptor on the surface of cancer cells conjugated to alliinase can deliver and target the enzyme alliinase to a tumor cell. Subsequent administration of alliin resulted in significant inhibition of the tumor growth.

There remains an unmet medical need for effective new and improved antifungal agents and protocols for the treatment of aspergillosis, including advanced stage disease. The present invention addresses these needs.

### SUMMARY OF THE INVENTION

The present invention provides monoclonal antibodies (mAbs) being capable of binding several members of the Aspergillus genus. The present invention further provides conjugates and fusion proteins of the anti-aspergillus monoclonal antibodies with the enzyme alliinase that upon interaction with the substrate alliin, produce the cytotoxic molecule allicin. The invention further provides pharmaceutical compositions comprising the mAbs or conjugates or fusion proteins thereof, in particular with alliinase. The invention further provides uses of the antibodies, conjugates to fusion proteins, alone or in combination with the substrate alliin for protection from or treatment of diseases cause by Aspergillus species, in particular invasive aspergillosis, and in increasing the survival rate of a subject infected with an Aspergillus species.

The present invention is based in part on the finding that mAbs against aspergillus fumigatus, and conjugates thereof with alliinase, were effective against pulmonary aspergillosis in vivo. These antibodies were shown to be selective to the aspergillus genus, namely, they react with at least some of the species of the to aspergillus genus but do not recognize other genera of fungi.

According to one aspect, the present invention provides an isolated monoclonal antibody against aspergillus, or an antibody fragment comprising at least an antigen-binding portion thereof, produced by a hybridoma cell which has been deposited with COLLECTION NATIONALE DE CULTURES DE MICROORGANISMES (CNCM) under Accession Number CNCM I-4377.

According to some embodiments the monoclonal antibody or fragment thereof comprises an antigen recognition domain having the complementarity determining region (CDR) sequences and orientation of the antibody produced from the hybridoma cell.

According to yet another aspect, the present invention provides monoclonal antibodies or fragments thereof comprising at least an antigen-binding portion thereof, that bind the same epitope as an antibody produced by a hybridoma cell which has been deposited under Accession Number CNCM I-4377.

According to some embodiments, a monoclonal antibody or a fragment thereof according to the invention comprises CDR residues from an antibody produced by a hybridoma cell which has been deposited under Accession Number CNCM I-4377.

According to some embodiments, a monoclonal antibody or a fragment thereof according to the invention comprises at least two CDR sequences selected from the group consisting of: CDR_{H1} (SEQ ID NO:1), CDR_{H2} (SEQ ID NO:2), CDR_{H3} (SEQ ID NO:14), CDR_{L1} (SEQ ID NO:3), CDR_{L2} (SEQ ID NO:4), and CDR_{L3} (SEQ ID NO:5).

According to other embodiments, a monoclonal antibody or a fragment thereof according to the invention comprises at least three CDR sequences selected from the group consisting of: CDR_{H1} (SEQ ID NO:1), CDR_{H2} (SEQ ID NO:2), CDR_{H3} (SEQ ID NO:14), CDR_{L1} (SEQ ID NO:3), CDR_{L2} (SEQ ID NO:4), and CDR_{L3} (SEQ ID NO:5).

According to yet other embodiments, a monoclonal antibody or a fragment thereof according to the invention comprises at least four CDR sequences selected from the group consisting of: CDR_{H1} (SEQ ID NO:1), CDR_{H2} (SEQ ID NO:2), CDR_{H3} (SEQ ID NO:14), CDR_{L1} (SEQ ID NO:3), CDR_{L2} (SEQ ID NO:4), and CDR_{L3} (SEQ ID NO:5).

According to yet other embodiments, a monoclonal antibody or a fragment thereof according to the invention comprises at least five CDR sequences selected from the group consisting of: CDR_{H1} (SEQ ID NO:1), CDR_{H2} (SEQ ID NO:2), CDR_{H3} (SEQ ID NO:14), CDR_{L1} (SEQ ID NO:3), CDR_{L2} (SEQ ID NO:4), and CDR_{L3} (SEQ ID NO:5).

According to one embodiment, the monoclonal antibody of the invention comprises a heavy chain variable region comprising the amino acid sequences of CDR_{H1} (SEQ ID NO:1) and CDR_{H2} (SEQ ID NO:2). According to another embodiment, the monoclonal antibody comprises a kappa light chain variable region comprising the amino acid sequences of CDR_{L1} (SEQ ID NO:3) and CDR_{L2} (SEQ ID NO:4). According to another embodiment, the monoclonal antibody comprises a heavy chain variable region comprising the amino acid sequences of CDR_{H1} (SEQ ID NO:1) and CDR_{H2} (SEQ ID NO:2) and a Kappa light chain variable region comprising the amino acid sequences of CDR_{L1} (SEQ ID NO:3), CDR_{L2} (SEQ ID NO:4).

According to a specific embodiment, the monoclonal antibody or a fragment thereof according to the invention comprises the CDR sequences: CDR_{H1} (SEQ ID NO:1), CDR_{H2} (SEQ ID NO:2), CDR_{H3} (SEQ ID NO:14), CDR_{L1} (SEQ ID NO:3), CDR_{L2} (SEQ ID NO:4), and CDR_{L3} (SEQ ID NO:5).

According to some embodiments, a monoclonal antibody or a fragment thereof according to the invention comprises a light chain variable region which comprises the amino acid sequence of SEQ ID NO:6.

According to other embodiments, a monoclonal antibody or a fragment thereof according to the invention comprises a heavy chain variable region which comprises the amino acid sequence of SEQ ID NO:7.

According to yet other embodiments, a monoclonal antibody or a fragment thereof according to the invention comprises a light chain variable region which comprises the amino acid sequence of SEQ ID NO:6, and a heavy chain variable region which comprises the amino acid sequence of SEQ ID NO:7, or an analog having at least 90% sequence identity with said variable region sequences.

According to further embodiments, analogs of the antibodies or fragments of the invention having at least 90%, 95% or 97% sequence identity with the sequences of said antibodies or fragments are disclosed.

According to some specific embodiments, a monoclonal antibody or a fragment thereof according to the invention is selected from the group consisting of:
(a) a monoclonal antibody having a heavy chain variable region comprising the amino acid sequences of CDR_{H1} (SEQ ID NO:1) and CDR_{H2} (SEQ ID NO:2);
(b) a monoclonal antibody having a Kappa light chain variable region comprising the amino acid sequences of CDR_{L1} (SEQ ID NO:3) and CDR_{L2} (SEQ ID NO:4);
(c) a monoclonal antibody having a heavy chain variable region comprising the amino acid sequences of CDR_{H1} (SEQ ID NO:1) and CDR_{H2} (SEQ ID NO:2); and a Kappa light chain variable region comprising the amino acid sequences of CDR_{L1} (SEQ ID NO:3) and CDR_{L2} (SEQ ID NO:4).

According to certain embodiments an antibody fragment comprising at least an antigen-binding portion of the antibody is provided. According to some embodiments the antibody fragment is selected from the group consisting of: a single chain antibody, an F(ab′)₂ fragment, F(ab) and Fv.

According to certain embodiments the isolated monoclonal antibody or fragment thereof according to the invention, is a natural, recombinant, chimeric or humanized antibody. According to some embodiments a monoclonal antibody or a fragment thereof according to the invention is of an immunoglobulin class selected from the group consisting of: IgG, IgM, IgE and IgA, and any subclass thereof. According to some embodiments the monoclonal antibody or fragment thereof is a humanized IgG antibody.

According to some embodiments, the antibodies of the invention recognize a common conserved epitope of the Aspergillus genus.

According to some embodiments, the antibodies of the invention recognize a carbohydrate structure on the surface of the Aspergillus.

Antibodies according to the present invention are selective, according to some embodiments, toward at least several species of the Aspergillus genus of fungi. According to these embodiments, the binding affinity of the monoclonal antibody towards members of the Aspergillus genus is at least one order of magnitude higher than its binding affinity to genera of fungi other than Aspergillus.

According to some embodiments the binding affinity of the monoclonal antibody of the invention towards Aspergillus species is at least one order of magnitude higher than its binding affinity towards the Candida species.

According to the present invention, a monoclonal antibody or a fragment thereof binds at least one Aspergillus species selected from the group consisting of Aspergillus fumigatus, Aspergillus niger, Aspergillus flavus, Aspergillus terreus, Aspergillus caesiellus, Aspergillus candidus, Aspergillus carneus, Aspergillus chevalieri, Aspergillus clavatus, Aspergillus deflectus, Aspergillus flavipes, Aspergillus glaucus, Aspergillus granulosus, Aspergillus nidulans, Aspergillus ochraceus, Aspergillus oryzae, Aspergillus parasiticus, Aspergillus penicilloides, Aspergillus restrictus, Aspergillus sojae, Aspergillus sydowi, Aspergillus tamari, Aspergillus ustus, Aspergillus versicolor and Aspergillus wentii.

According to some embodiments, a monoclonal antibody or a fragment thereof binds a plurality of Aspergillus species selected from the group consisting of Aspergillus fumigatus, Aspergillus niger, Aspergillus flavus, Aspergillus terreus, Aspergillus caesiellus, Aspergillus candidus, Aspergillus carneus, Aspergillus chevalieri, Aspergillus clavatus, Aspergillus deflectus, Aspergillus flavipes, Aspergillus glaucus, Aspergillus granulosus, Aspergillus nidulans, Aspergillus ochraceus, Aspergillus oryzae, Aspergillus parasiticus, Aspergillus penicilloides, Aspergillus restrictus, Aspergillus sojae, Aspergillus sydowi, Aspergillus tamari, Aspergillus ustus, Aspergillus versicolor and Aspergillus wentii.

According to some embodiments, the antibody of the invention or a fragment thereof specifically binds Aspergillus species selected from the group consisting of Aspergillus fumigatus, Aspergillus niger, Aspergillus flavus and Aspergillus terreus.

According to yet other embodiments the antibody of the invention or a fragment thereof specifically binds the Aspergillus species Aspergillus fumigatus, Aspergillus niger, Aspergillus flavus and Aspergillus terreus.

According to some currently preferred embodiments, the antibody of the invention specifically binds Aspergillus fumigatus.

According to some embodiments, the antibodies of the invention are protective against Aspergillus species. According to some other embodiments, the antibodies of the invention confer protection to a subject against Aspergillus species. According to one embodiment, the subject is a mammal. According to another embodiment, the mammal is a human.

Fragments of the antibodies according to the present invention comprise at least an antigen-binding portion of the antibody. These fragments are also denoted “antigen recognition fragments”. An antigen recognition fragment according to the invention is selected, according to some embodiments, from the group consisting of a single chain antibody and an antibody fragment including, but not limited to, an F(ab′)₂ fragment, F(ab) or Fv. Each possibility represents a separate embodiment of the invention.

An isolated monoclonal antibody according to the invention, or a fragment, conjugate or fusion protein thereof is of either human or animal origin. It may be a natural, recombinant or humanized antibody, or it may be a chimeric antibody. A monoclonal antibody according to the invention or a fragment, conjugate or fusion protein thereof may be of any immunoglobulin class including IgG, IgM, IgE, IgA, and any subclass thereof.

According to some embodiments the monoclonal antibody according to the invention, or the fragment, conjugate or fusion protein thereof is a humanized IgG antibody.

The present invention provides, according to another aspect, conjugates and fusion proteins comprising at least one monoclonal antibody against aspergillus, or a fragment thereof comprising at least an antigen-binding portion of the antibody.

According to some embodiments a conjugate or fusion protein of the antibody or antibody fragment of the invention with an enzymatically active form of alliinase is provided. According to one embodiment, the alliinase in the conjugate or fusion protein is being able to generate allicin or analogues thereof from alliin substrates or analogues thereof. According to some embodiments, the alliinase is a natural alliinase. According to some specific embodiments, the alliinase is garlic alliinase. According to some further embodiments, the alliinase is pegylated.

According to some embodiments of the present invention, the alliinase and the monoclonal antibodies of the invention are linked by chemical means by generating covalently bound conjugates. According to certain exemplary embodiments, the antibody of the invention is chemically ligated to the enzyme alliinase.

According to other embodiments, the antibody or fragment of the invention is part of a recombinantly expressed fusion protein. According to some embodiments, the fusion protein comprises an antibody or fragment according to the invention and alliinase. According to some embodiments the fusion protein is expressed as a single polypeptide comprising at least the antigen binding portion of an antibody of the invention and the enzyme alliinase.

The antibody or antigen binding fragment thereof and the enzyme alliinase may be linked, in a conjugate or in a fusion protein, through a linker. According to some embodiments the linker is a peptide linker. According to some embodiments, the linker comprises up to 30 amino acid residues. Alternatively, the linker comprises up to 25 amino acid residues, up to 20 amino acid residues, up to 15 amino acid residues, up to 10 amino acid residues, or up to 5 amino acid residues. Each possibility represents a separate embodiment of the present invention. According to some embodiments, the peptide linker is a flexible linker.

According to yet other embodiments the enzyme and the monoclonal antibody are associated by physical means such as by affinity binding, by entrapping the enzyme within a polymer matrix or membrane (liposome), or microencapsulating the enzyme within semipermeable polymer membranes.

According to another aspect, the present invention provides isolated polynucleotides encoding the monoclonal antibody or fragment of the invention or a fusion protein according to the invention. The polynucleotide may encode the whole antibody or the light chain variable region or the heavy chain variable region or both chains of the variable region of the antibody.

The invention further provides vectors comprising polynucleotides encoding the antibody of the invention or fragments thereof. Consequently, the antibody of the invention may be expressed in a host cell following co-transfection of the heavy and light chain vectors or by transfection of a single vector comprising both light and heavy chain polynucleotide sequences.

According to another embodiment, the present invention provides an isolated polynucleotide encoding the kappa light chain variable region of an antibody, fragment or fusion protein according to the invention, wherein the kappa light chain variable region comprises the amino acid sequence as set forth in SEQ ID NO. 6.

According to another embodiment, the polynucleotide encoding the kappa light chain of the antibody, fragment or fusion protein of the invention comprises the nucleotide sequence as set forth in SEQ ID NO. 8.

According to another embodiment, the present invention provides an isolated polynucleotide encoding the heavy chain variable region of an antibody, fragment or fusion protein of the invention, wherein the heavy chain variable region comprises the amino acid sequence as set forth in SEQ ID NO. 7.

According to yet other embodiments, the present invention provides an isolated polynucleotide encoding the heavy chain variable region of an antibody, fragment or fusion protein of the invention, wherein the heavy chain variable region comprises the amino acid sequence as set forth in SEQ ID NO. 15.

According to yet another embodiment, the isolated polynucleotide encoding the heavy chain of an antibody, fragment or fusion protein of the invention comprises the nucleotide sequence as set forth in SEQ ID NO. 9.

According to some embodiments, the present invention provides isolated polynucleotides encoding a fusion protein comprising at least the antigen binding portion of a monoclonal antibody of the invention and the enzyme alliinase. The polynucleotide may encode intact chains of the antibody and alliinase or at least the antigen binding portions thereof or a single chain variable fragment of the antibody and alliinase.

According to yet another embodiment, the present invention provides a vector comprising the polynucleotide sequence encoding the antibody, fragment or fusion protein of the invention. According to yet another embodiment, the present invention provides a vector comprising the polynucleotide sequence encoding the antibody, fragment or fusion protein of the invention selected from the group consisting of whole antibody, the light chain variable region, the heavy chain variable region, both chains of the variable region. Each possibility represents a separate embodiment of the invention.

According to some embodiments, the vector comprises the polynucleotide sequence encoding an antibody of the invention or fragments thereof and alliinase.

According to yet another embodiment, the present invention provides a vector comprising the polynucleotide sequence encoding the antibody of the invention or fragments thereof selected from the group consisting of whole antibody, the light chain variable region, the heavy chain variable region, both chains of the variable region and alliinase. Each possibility represents a separate embodiment of the invention.

According to yet another embodiment, the present invention provides a vector comprising a polynucleotide sequence encoding the kappa light chain variable region of the antibody of the invention, wherein the kappa light chain variable region comprises the amino acid sequence as set forth in SEQ ID NO. 6. According to yet another embodiment, the vector comprises a polynucleotide sequence comprising the nucleotide sequence as set forth in SEQ ID NO. 8.

According to yet another embodiment, the present invention provides a vector comprising a polynucleotide sequence encoding the heavy chain variable region of the antibody of the invention, wherein the heavy chain variable region comprises the amino acid sequence as set forth in SEQ ID NO. 7. According to yet another embodiment, the vector comprises a polynucleotide sequence comprising the nucleotide sequence as set forth in SEQ ID NO. 9.

According to some embodiments vectors comprising polynucleotides encoding the fusion protein of the antibody of the invention or fragments thereof and alliinase are provided.

According to some embodiments, the vector may further comprise at least one polynucleotide sequence encoding a component selected from the group consisting of: a promoter operably linked to the polynucleotide encoding the antibody, one or more resistance gene, a Kozak sequence, an origin of replication, one or more selection marker genes, an enhancer element, transcription terminator, a signal peptide, genomic human kappa constant region and genomic human IgG constant region.

According to some other embodiments, the vector may be a plasmid or a virus.

According to another aspect, the present invention provides cells containing a vector comprising the polynucleotide sequence encoding an antibody of the invention or fragment or fusion protein thereof for the purposes of storage, propagation, antibody and fusion protein production and therapeutic applications.

According to some embodiments, the cell comprises a vector comprising a polynucleotide sequence encoding an antibody of the invention or fragment thereof and alliinase.

According to another aspect, the present invention provides a host cell comprising the vector according to the embodiments of the invention as depicted above, wherein the host cell is capable of expressing the antibody of the invention or fragments or fusion protein thereof. According to some embodiments, the host cell is selected from eukaryotic host cell and prokaryotic host cell.

According to some embodiments the host cell is capable of expressing a fusion protein comprising the antibody of the invention or fragments thereof and alliinase. According to some specific embodiments, the host cell is a plant cell.

The fusion protein of the antibody of the invention or fragments thereof and alliinase may be expressed in a host cell following co-transfection of the heavy and light chain vectors and alliinase or by transfection of a single vector comprising both light and heavy chain polynucleotide sequences and alliinase.

According to yet another aspect, the present invention provides a hybridoma cell line capable of producing the antibody of the invention or fragments thereof. According to one embodiment, the hybridoma cell line is AF293-MPS 5.44, deposited under Accession No. CNCM I-4377.

According to yet another aspect, the present invention provides a pharmaceutical composition comprising at least one monoclonal antibody, a fragment thereof, a conjugate or a fusion protein, according to embodiments of the invention and a pharmaceutically acceptable carrier.

According to some embodiments the pharmaceutical composition comprises an unconjugated antibody against Aspergillus or a fragment thereof and a pharmaceutically acceptable carrier.

According to some embodiments the pharmaceutical composition comprises a conjugate or fusion protein of an antibody or fragment according to various embodiments of the invention, with the enzyme alliinase, and a pharmaceutically acceptable carrier. According to these embodiments, the conjugate or fusion protein is use for guiding the enzyme to an Aspergillus species in the body, wherein the activity of the alliinase and antibody is preserved in the conjugate.

Pharmaceutical compositions according to the present invention can be administered by any convenient route, including, intratracheal instillation, inhalation, oral, intranasal, parenteral, subcutaneous, intravenous, intramuscular, intraperitoneal, or transdermal. According to some currently preferred embodiments, the pharmaceutical composition is formulated for inhalation and/or intratracheal instillation.

The present invention provides according to yet another aspect, methods for protecting against and treating of diseases and disorders cause by Aspergillus species, comprising administering to the subject in need thereof a pharmaceutical composition comprising at least one antibody, fragment, conjugate or fusion protein thereof according to the invention.

According to some embodiments the method of treating comprises administering the pharmaceutical composition at an advanced stage of Aspergillus infection.

According to one embodiment, a method for increasing the survival rate of a subject infected with an Aspergillus species is provided, comprising administering to the subject a monoclonal antibody according to embodiments of the invention or antigen binding fragments thereof. According to some embodiments, the antibodies of the invention inhibit the growth or survival of Aspergillus species.

According to other embodiments, the present invention provides a method for treating diseases or conditions caused by Aspergillus in a subject comprising administering to the subject a pharmaceutical composition comprising at least one antibody against Aspergillus or a fragment, conjugate or fusion protein thereof.

According to some embodiments, the method comprises administering to a subject a conjugate or a fusion protein of the enzyme alliinase with the monoclonal antibody of the invention or antigen binding fragments thereof followed by administration of alliin. The conjugates or fusion proteins of the enzyme alliinase with the monoclonal antibodies of the invention target the alliinase to an Aspergillus cell and are used in combination with alliin to produce allicin at the Aspergillus cell, thus killing the cell.

According to some embodiments, alliin is administered after the administration of the conjugate or fusion protein of the invention. According to some other embodiments, alliin may be administered not more than 24 hours after the administration of the conjugate or fusion protein; alternatively, not more than 12 hours after the administration of the conjugate or fusion protein; alternatively, not more than 6 hours; alternatively, not more than 3 hours; alternatively, not more than 2 hours; alternatively, not more than 1 hour; alternatively, not more than 30 minutes after the administration of the conjugate or fusion protein.

The conjugate or fusion protein and the alliin may be administered using same or different modes of administration. For example, both the conjugate and alliin may be administered intratracheally; alternatively, the conjugate may be administered intratracheally while alliine may be administered orally or intravenously. According to some embodiments, the administration of alliin may be repeated one or several times as necessary after the administration of the conjugate, in intervals to be decided according to the stage of the disease and condition of the patient.

According to yet another aspect, the present invention provides a method for treating diseases or conditions caused by Aspergillus in a subject comprising administering to the subject an unconjugated monoclonal antibody according to various embodiments of the invention or antigen binding fragments thereof.

According to some embodiments, the methods of the invention are useful for the treatment of diseases or conditions caused by Aspergillus, the diseases or conditions are selected from the group consisting of invasive aspergillosis, allergic bronchopulmonary aspergillosis, chronic necrotizing aspergillus pneumonia and pulmonary aspergilloma. According to currently preferred embodiments, the methods of the invention are useful for the treatment of invasive aspergillosis.

Use of the monoclonal antibodies, antibody fragments, conjugates and fusion proteins according to the invention for preparation medicaments for protecting against or treating against diseases or conditions caused by Aspergillus is also within the scope of the present invention.

Another aspect of the present invention provides use of antibodies or fragments thereof for detecting Aspergillus species in vitro, ex vivo or in vivo.

These and other features and advantages of the present invention will become more readily understood and appreciated from the detailed description of the invention that follows.

### BRIEF DESCRIPTION OF THE FIGURES

FIG. 1 shows the Size-exclusion chromatography of IgM-Alliinase conjugate preparation (MPS 5.44-Alliinase) on a Superdex 200 column. Solid line: absorption at 280 nm. Dotted line: alliinase units of activity (μmol/min⁻¹/ml⁻¹). The conjugate+IgM is represented by the first peak (˜120 min); alliinase is represented by the second peak (˜180 min) and N-ethyl maleimide that had been added to stop the reaction of conjugation is represented by the third peak (˜280 min). Fractions of the first peak with the highest alliinase activity were pooled and used as the purified conjugate.

FIG. 2 shows the binding of the antibody and conjugates to A. fumigatus. (A) ELISA binding curves to hyphae of A. fumigatus. (⋄)—Conjugates mAb(MPS5.44)-alliinase; (□)— antibody MPS 5.44; (x)—non-specific mouse IgM; (Δ)—conjugate of non-specific IgM with alliinase. (B) Conjugate mAb(MPS5.44)-alliinase bound efficiently to all forms of AF, i.e. (□)—swollen conidia, (⋄)— resting conidia as well as (Δ)—hyphae. Binding of a conjugate of a non-specific mouse IgM-alliinase (dashed lines) to all forms of AF was negligible: (x)—swollen, (◯)—resting conidia and ()—hyphae. (C) Comparison between the binding of FITC-labeled conjugate (upper panel) and alliinase (lower panel) to hyphae of A. fumigatus CBS144.89/DsRed. (D) Species specificity of mAb MPS 5.44. Binding curves of mAb to hyphae of different fungi as determined by ELISA (⋄) A. fumigatus 293, (□)—A. flavus, (◯)— A. niger, (Δ)—A. terreus 9, (▴)—C. krusei, and (*)—C. albicans.

FIG. 3 shows the alliinase activity of alliinase conjugates bound to A. fumigatus conidia. (A) Saturation of the conidia with (i) MPS5.44-alliinase conjugates (light grey), (ii) a non-specific IgM-alliinase conjugate (white) or (iii) free alliinase (dark gray). Specific binding of MPS5.44-alliinase conjugates to conidia reached saturation in 20 min. Longer incubation did not increase the binding of the conjugate; (B) alliinase activity of the MPS5.44-alliinase conjugates was preserved on the surface of conidia for at least 3 hours.

FIG. 4 shows the binding of the antibody to the hyphae (A) or fixed hyphae (B) of Aspergillus fumigatus pretreated with sodium periodate. The antibody of the invention binds Aspergillus fumigatus at concentrations as low as 1 nM, however periodic oxidation disturb the binding completely. (⋄) Hyphae treated with PBS; (□) hyphae treated with sodium periodate. Binding is expressed as the mean OD₄₀₅ spectrophotometric readings from triplicate wells after subtraction of the absorption from wells containing Aspergillus fumigatus in the presence of non-specific monoclonal antibodies.

FIG. 5 shows the antifungal activity of the conjugates in the presence of alliin. (A) Inhibition of fungal growth (swollen conidia) by mAb(MPS5.44)-alliinase conjugate and alliin (⋄); mAb(MPS5.44)-alliinase conjugate without alliin (□); unconjugated mAb (MPS5.44) (▴); non-conjugated alliinase and alliin (◯); conjugate of non-specific mAb IgM-alliinase and alliin (Δ); (B) Shows the minimal fungicidal concentration (MFC) of the mAb(MPS5.44)-alliinase conjugate (light gray) or of unconjugated alliinase (dark gray) for resting conidia, swollen conidia and hyphae.

FIG. 6 shows the hyphal growth inhibitory activity by the conjugates. (A)—Control sample in the absence of antibody; (B) in the presence of 20 nmol unconjugated mAb MPS5.44; (C) in the presence of 20 nmol mAb(MPS5.44)-alliinase conjugate without alliin; (D) 10 pmol of mAb(MPS5.44)-alliinase conjugate with alliin (0.5 mg/ml).

FIG. 7 shows the reduction in lung fungal load in AF-infected mice. Upper panel—Confocal microscopic analysis of the lungs (A-D). (A) a representative picture (from one mouse out of 5) of PBS/alliin treated mice; (B) a representative picture (one mouse out of 5) of mAb-alliinase conjugate/alliin treated mice; (C) A representative picture of a PBS-treated mouse taken at day 13 when the fungus spread throughout the lungs; (D) a representative picture of the lungs of a mouse that was treated four times with mAb-alliinase conjugate/alliin at day 36. Lower panel—Histological examination of lung sections of infected mice (E-H). (E,F) show representative lung sections taken from mice which died during the second week of infection as a result of severe necrotizing bronchitis with invasion of vascular walls by fungal hyphae. (G,H) Mice treated four times with mAb-alliinase conjugate+alliin, survived and their lungs had normal airway epithelium.

FIG. 8 discloses the survival rate of infected mice. () PBS-treated mice; (▪)—PBS+alliin treated mice; (Δ) mice treated with mAb-alliinase conjugate with PBS (no alliin); (▾) unconjugated mAb (MPS 5.44)+PBS; (◯) mice treated with mAb-alliinase conjugate+alliin (treatment started on day 0); (⋄) mice treated with non-conjugated alliinase+alliin; (□) mice treated with mAb-alliinase conjugate+alliin (treatment started on day 2).

### DETAILED DESCRIPTION OF THE INVENTION

The present invention provides monoclonal antibodies specific for members of the Aspergillus genus and uses thereof for increasing the survival rate of a subject infected with an Aspergillus species.

Monoclonal antibodies specific for members of the Aspergillus genus are now found to have considerable activity against the fungus, displaying significant prolonged survival in an animal model of pulmonary aspergillosis. The invention thus provides pharmaceutical compositions comprising the mAbs of the invention or antigen binding fragments thereof and uses thereof in the treatment of diseases cause by Aspergillus species, in particular invasive aspergillosis.

The present invention further provides conjugates of the enzyme alliinase with monoclonal antibodies specific for members of the Aspergillus genus that preserve the ability of the antibody to recognize the fungus as well as the activity of the enzyme to produce cytotoxic molecules of allicin from the substrate alliin. The invention further provides pharmaceutical compositions comprising the conjugates of the mAbs of the invention or antigen binding fragments thereof and alliinase and uses thereof in combination with the substrate alliin in the treatment of diseases cause by Aspergillus species, in particular invasive aspergillosis.

The present invention is based in part on the finding that significantly high survival rates of about 85% with almost complete fungal clearance were observed in an animal model of pulmonary aspergillosis upon administration of conjugates of alliinase and a monoclonal antibody raised against aspergillus fumigatus followed by administration of alliin. Interestingly, administrations of the conjugate and alliin were also highly effective when treatment was initiated at an advanced stage of infection (50 hours post infection).

Conjugates and fusion proteins of the present invention enable the in vivo delivery of allicin to the pathogenic aspergillus species found in the body of a subject, where allicin should exert its desired biological activity (i.e. kill the aspergillus cells). By using the conjugates of the invention in the presence of alliin, the hydrophobic allicin molecules are produced on or in the vicinity of the aspergillus cell and thus exert only a limited local effect. Furthermore, due to their high reactivity and short lifetime, the allicin molecules kill the fungus without causing visible damage to adjacent cells.

Monoclonal antibodies according to the invention, raised against aspergillus fumigatus are cross reactive with other aspergillus species such as aspergillus niger, aspergillus flavus and aspergillus terreus, but do not recognize other genera of fungi.

The present invention provides monoclonal antibodies having specific binding affinity to Aspergillus. It is to be emphasized, that the monoclonal antibodies of the invention were raised against a single Aspergillus species, i.e. Aspergillus fumigatus, yet they cross react with other Aspergillus species, thus display specific binding affinity towards other Aspergillus species. According to one embodiment, the antibodies of the invention do not cross react with other fungal genus such as Candida. According to another embodiment, the antibodies of the invention do not cross react with mammalian cells.

By “specific binding affinity” it is meant that the antibody of the invention binds to the target Aspergillus cells with greater affinity than it binds to other fungal cells and/or mammalian cells under specified conditions. According to one embodiment, the antibody of the invention binds the target Aspergillus cells with an affinity greater by at least one order of magnitude than it's binding affinity to other fungal cells and/or mammalian cells under specified conditions. Typically, an antibody according to the present teachings is capable of binding at least one species of Aspergillus with a minimal affinity of 10⁻⁸ or 10⁻⁹ M.

The monoclonal antibodies according to embodiments of the invention may be referred to as protective antibodies. The term “protective antibody” designates antibodies capable of conferring protection against an Aspergillus species in a human or animal host infected with an Aspergillus species.

The present invention provides a method to generate allicin in vivo on an Aspergillus cell, wherein allicin is generated directly at the Aspergillus cell or in the vicinity of said Aspergillus cell so that the allicin does not deteriorate prior to reaching the target cell. The allicin-producing enzyme, alliinase, is delivered directly to the sites where allicin is desired to produce its effect. Following administration of the biologically inactive and non-toxic alliinase substrate alliin, allicin production occurs only at the place where the alliinase is located. The advantages of this approach are based on-the specific features of allicin, namely, its potent biological activity, its ability to rapidly penetrate through biological membranes, its extremely short lifetime in the body, and its conversion into non-toxic and even beneficial secondary products. Thus, according to the present invention, the biologically potent active molecule allicin is generated by the alliin-alliinase system at the or in the vicinity of Aspergillus cells, and thus allicin will exert its toxic effect locally.

The present invention uses the site-directed in situ production of allicin to combat microbial diseases. According to currently preferred embodiment, the microbial diseases are microbial diseases of the respiratory system.

The present invention provides the enzyme alliinase in an enzymatically active form conjugated with the antibody of the invention which serves as a targeting carrier which guides the enzyme to aspergillus species found in the body.

### Aspergillus

Aspergillus is a filamentous, cosmopolitan and ubiquitous fungus found in nature. The genus Aspergillus includes over 185 species. Around 20 species have so far been reported as causative agents of opportunistic infections in man. Among these, Aspergillus fumigatus is the most commonly isolated species, followed by Aspergillus flavus, Aspergillus niger, Aspergillus clavatus, Aspergillus glaucus group, Aspergillus nidulans, Aspergillus oryzae, Aspergillus terreus, Aspergillus ustus, and Aspergillus. Aspergillus may cause a broad spectrum of disease in the human host, ranging from hypersensitivity reactions to direct angioinvasion. Aspergillus primarily affects the lungs, causing 4 main syndromes, including allergic bronchopulmonary aspergillosis (ABPA), chronic necrotizing Aspergillus pneumonia (or chronic necrotizing pulmonary aspergillosis (CNPA)), aspergilloma, and invasive aspergillosis. However, in patients who are severely immunocompromised, Aspergillus may hematogenously disseminate beyond the lung, potentially causing endophthalmitis, endocarditis, and abscesses in the myocardium, kidney, liver, spleen, soft tissue, and bone. Aspergillus is second to Candida species as a cause of fungal endocarditis. Aspergillus-related endocarditis and wound infections may occur for example, during cardiac surgery.

Allergic bronchopulmonary aspergillosis (ABPA) is a hypersensitivity reaction to Aspergillus fumigatus colonization of the tracheobronchial tree and occurs in conjunction with asthma and cystic fibrosis (CF). Allergic fungal sinusitis may also occur alone or with ABPA. Bronchocentric granulomatosis and malt worker's lung are two hypersensitivity lung diseases that are caused by Aspergillus species.

An aspergilloma is a fungus ball (mycetoma) that develops in a preexisting cavity in the lung parenchyma. Underlying causes of the cavitary disease may include treated tuberculosis as well as other necrotizing infection, sarcoidosis, CF, and emphysematous bullae. The ball of fungus may move within the cavity but does not invade the cavity wall, yet it may cause hemoptysis.

Chronic necrotizing pulmonary aspergillosis (CNPA) is a subacute process usually found in patients with some degree of immunosuppression, most commonly that associated with underlying lung disease, alcoholism, or long-term corticosteroid therapy. CNPA often remains unrecognized for weeks or months and can cause a progressive cavitary pulmonary infiltrate.

Invasive aspergillosis is a rapidly progressive, often fatal infection that occurs in patients who are severely immunosuppressed, including those who are profoundly neutropenic, those who have received bone marrow or solid organ transplants, and patients with advanced AIDS or chronic granulomatous disease. This infectious process is characterized by invasion of blood vessels, resulting in multifocal infiltrates, which are often wedge-shaped, pleural-based, and cavitary. Dissemination to other organs, particularly the central nervous system, may occur.

### Antibodies

The monoclonal antibody according to various embodiments of the invention is of either human or animal origin. It may be a natural, recombinant or humanized antibody, or it may be a derivative thereof such as a F(ab)₂ dimer or F(ab) monomer, Fv or a natural or recombinant single-chain Fv. The antibody must be one that recognizes a specific feature (e.g., a receptor, a carbohydrate structure) on the microbial cell surface to be targeted and are not internalized. Without wishing to be bound by theory or mechanism of action it is suggested that the antibodies of the invention recognized the carbohydrate structure of Aspergillus as depicted by the lack of binding of the antibody of the invention to Aspergillus treated with periodate. Antigen recognition fragments also referred to as monoclonal antibody derivatives are those molecules that recognize the same feature on the cell surface as the monoclonal antibody and thus can be used interchangeably with the intact monoclonal antibody in the present invention.

It should be understood that when the term “antibodies” is used with respect to the antibody of the present invention, this is intended to include intact antibodies, such as polyclonal antibodies or monoclonal antibodies (mAbs), as well as proteolytic fragments thereof such as the Fab or F (ab)₂ fragments. Each possibility represents a different embodiment of the present invention. Furthermore, the DNA encoding the variable region of the antibody can be inserted into other antibodies to produce chimeric antibodies (see, for example, U.S. Pat. No. 4,816,567) or into T-cell receptors to produce T-cells with the same broad specificity (see Eshhar et al, 1990 and Gross et al, 1989).

Single-chain antibodies can also be produced and used. Single chain antibodies can be single-chain composite polypeptides having antigen binding capabilities and comprising a pair of amino acid sequences homologous or analogous to the variable regions of an immunoglobulin light (VL) and heavy chain (VH) (linked VH-VL or single-chain FV). Both VH and VL may copy natural monoclonal antibody sequences or one or both of the chains may comprise a complementary determining region (CDR)-framework region (FR) construct of the type described in U.S. Pat. No. 5,091,513 (the entire content of which is hereby incorporated herein by reference). The separate polypeptides analogous to the variable regions of the light and heavy chains are held together by a polypeptide linker.

Methods of production of such single-chain antibodies, particularly where the DNA encoding the polypeptide structures of the VH and VL chains are known, may be accomplished in accordance with the methods described in, for example, U.S. Pat. Nos. 4,946,778; 5,096,815 and 5,091,513.

An antibody is said to be “capable of binding” a molecule if it is capable of interacting specifically with the molecule and wherein said interaction may result with the binding of the molecule to the antibody. The term “epitope” is meant to refer to that portion of any molecule capable of being bound by an antibody which can also be recognized by that antibody. Epitopes or “antigenic determinants” usually consist of chemically active surface groupings of molecules such as amino acids or sugar side chains and have specific three dimensional structural characteristics as well as specific charge characteristics.

Monoclonal antibodies (mAbs) are a substantially homogeneous population of antibodies to specific antigens. MAbs may be obtained by methods known to those skilled in the art. See, for example U.S. Pat. No. 4,376,110. Such antibodies may be of any immunoglobulin class including IgG, IgM, IgE, IgA, and any subclass thereof. The hybridoma producing the mAbs of this invention may be cultivated in vitro or in vivo. High titers of mAbs can be obtained by in vivo production where cells from the individual hybridomas are injected intraperitoneally into pristane-primed Balb/c mice to produce ascites fluid containing high concentrations of the desired mAbs. MAbs of isotype IgM or IgG may be purified from such ascites fluids, or from culture supernatants, using column chromatography methods well known to those of skill in the art.

Chimeric antibodies are molecules, the different portions of which are derived from different animal species, such as those having a variable region derived from a murine mAb and a human immunoglobulin constant region. Chimeric antibodies are primarily used to reduce immunogenicity during application and to increase yields in production, for example, where murine mAbs have higher yields from hybridomas but higher immunogenicity in humans, such that human/murine chimeric or humanized mAbs are used. Chimeric and humanized antibodies and methods for their production are well-known in the art, such as Cabilly et al, European Patent No. 0125023; Taniguchi et al, European Patent No. EP 0171496; Morrison et al, European Patent No. EP 0173494; Neuberger et al, international publication WO 86/01533; Kudo et al, European Patent No. EP0184187; Robinson et al, international publication WO 87/02671. These references are hereby incorporated herein by reference.

A “molecule which includes the antigen-binding portion (also referred to as antigen recognition fragment) of an antibody”, is intended to include not only intact immunoglobulin molecules of any isotype and generated by any animal cell line or microorganism, or generated in vitro, such as by phage display technology for constructing recombinant antibodies, but also the antigen-binding reactive fraction thereof (also referred to herein as a derivative thereof), including, but not limited to, the Fab fragment, the Fab′ fragment, the F(ab′)₂ fragment, the variable portion of the heavy and/or light chains thereof, and chimeric or single-chain antibodies incorporating such reactive fraction, or molecules developed to deliver therapeutic moieties by means of a portion of the molecule containing such a reactive fraction. Such molecules may be provided by any known technique, including, but not limited to, enzymatic cleavage, peptide synthesis or recombinant techniques.

Recombinant techniques: Antibodies can be generated in vitro using phage display technology or by the generation and application of very large standardized universal ‘singlepot’ antibody gene libraries, which in principle contain binders against every possible antigen.

Production of recombinant antibodies by such recombinant techniques is much faster compared to conventional antibody production and they can be generated against an enormous number of antigens. In contrast, in the conventional method, many antigens prove to be non-immunogenic or extremely toxic, and therefore cannot be used to generate antibodies in animals. Moreover, affinity maturation (i.e., increasing the affinity and specificity) of recombinant antibodies is very simple and relatively fast. Finally, large numbers of different antibodies against a specific antigen can be generated in one selection procedure. To generate recombinant monoclonal antibodies one can use various methods based on phage display libraries or by the generation and application of very large standardized universal singlepoe antibody gene libraries to generate a large pool of antibodies with different antigen recognition sites. Phage display libraries can be made in several ways: One can generate a synthetic repertoire by cloning synthetic CDR3 regions in a pool of heavy chain germline genes and thus generating a large antibody repertoire, from which recombinant antibody fragments with various specificities can be selected. One can use the lymphocyte pool of humans as starting material for the construction of an antibody library.

According to some embodiments of the present invention recombinant antibody comprising the gene coding for the scFv chain may be expressed using mammalian expression vectors, such as the ones derived from the pCMV vectors (e.g. pCMX2.5-hIgG1-Fc-XP) (Schutte, M., et al., (2009), PLoS One 4, e6625) or any other available vectors (some of which are described below for the production of antibody-alliinase recombinant conjugates) which enable the fusion of scFv antibodies to the gene fragment encoding the human IgG1 Fc part (hinge-CH₂—CH₃) and allows the production of homodimeric scFv-Fc fusion proteins in HEK293 T cells.

Allinase

Any alliinase or enzyme which has alliinase activity, either natural or recombinant (Rabinkov A. et al., Appl. Biochem. Biotechnol. (1994) 48:149-71), may be used in the present invention, whether the entire molecule or a derivative or fragment thereof, as long as it retains its catalytic activity and ability to generate allicin or analogs thereof in a lyase reaction from alliin substrates or analogs thereof. While natural alliinase from garlic is the preferred enzyme for use in the present invention, alliinase from any source, including onion, Brassicaceae, Fabaceae, broccoli, and even bacteria can be used. Representative amino acid sequences for alliin from a variety of plant sources include but are not limited to those of GenBank accession numbers S35460, S29302, S29300, S29301, BAB68045, BAB68042, AAK95663, AAK96552, AAK95661, AAK95660, AAK95659, AAK95698, AAK95657, AAK95656, NP177213, NP173746, P31757, AAG52476, AAG52348, AAG12844, AAG00599, AAF81248, AAF36437, Q01594, P31756, AAD51706, AAD51705, AAD51704, AAD51703, AAD51702, AAD51701, AAD43130, AAD32696, AAD26853, AAD21617, BAA20358, AAB32477, CAA78268, CAA78267, CAA78266, CAA63482, AAA92463, and AAA32639. Sequence analysis of alliinase cDNA clones from different Alliaceae species revealed a high degree of sequence similarity, both at the nucleotide and at the amino acid levels. However, changes in the sequences can be made as long as the alliinase activity of the protein is retained.

Identification of alliinase activity can be performed by any technique known in the art such as the techniques described by Miron et al., 1998, Analyt. Biochem. 265: 317-25, Jin et al., 2001, Bull. Korea Chem. Soc., 22(1):68-76, or by Manabe et al., 2000, ‘Sulfur nutrition and sulfur assimilation in higher plants’, Brunold et al (eds.), Paul Haupt, Berne, pp. 419-20. Any technique known in the art which is used to identify alliinase activity can be further used by one skilled in the art to determine if an analog, fragment, or derivative of alliinase or a related lyase has alliinase activity that can be used to convert alliin to allicin. If an enzyme has substantially the same type of activity on alliin as alliinase from garlic, that enzyme can be used in conjugates according to the present invention. For purposes of the present invention, “substantially the same activity” means that the enzyme fragment, derivative or analog has at least 50% of the activity of alliinase from garlic in converting alliin to allicin.

An analog of alliinase has an amino acid sequence essentially corresponding to any one of the amino acid sequences of alliinase available in GenBank disclosed above. The term “essentially corresponding to” is intended to comprehend analogs with minor changes to the sequence of the protein or polypeptide which do not affect the basic characteristics thereof, particularly insofar as its catalytic activity and ability to generate allicin or analogs thereof in a lyase reaction from alliin substrates or analogs thereof is concerned. The type of changes which are generally considered to fall within the “essentially corresponding to” language are those which would result from conventional mutagenesis techniques of the DNA encoding alliinase, resulting in a few minor modifications, and screening for the desired activity in the manner discussed above.

Preferably, the analog is a variant of a native sequence or a biologically active fragment thereof which has an amino acid sequence having at least 70% identity to a native amino acid sequence and retains the biological activity thereof. More preferably, such a sequence has at least 85% identity, at least 90% identity, or most preferably at least 95% identity to a native sequence.

Analogs in accordance with the present invention may also be determined in accordance with the following procedure. Polypeptides encoded by any nucleic acid, such as DNA or RNA, which hybridize to the complement of the native DNA or RNA under highly stringent or moderately stringent conditions, as long as that polypeptide maintains the alliinase catalytic activity of a known native sequence are also considered to be within the scope of the present invention.

The alliinase enzyme as used according to embodiments of the invention may further include functional derivatives of alliinase. “Functional derivatives” as used herein covers chemical derivatives which may be prepared from the functional groups which occur as side chains on the residues or the N- or C-terminal groups, by means known in the art, and are included in the invention as long as they remain pharmaceutically acceptable, i.e., they do not destroy the catalytic activity of the corresponding alliinase enzyme as described herein.

Derivatives may have chemical moieties, such as carbohydrate or phosphate residues, provided such a fraction has the same catalytic activity and remains pharmaceutically acceptable.

Suitable derivatives may include aliphatic esters of the carboxyl of the carboxyl groups, amides of the carboxyl groups by reaction with ammonia or with primary or secondary amines, N-acyl derivatives or free amino groups of the amino acid residues formed with acyl moieties (e.g., alkanoyl or carbocyclic aroyl groups) or O-acyl derivatives of free hydroxyl group (e.g., that of seryl or threonyl residues) formed with acyl moieties. Such derivatives may also include for example, polyethylene glycol side-chains which may mask antigenic sites and extend the residence of the complex or the portions thereof in body fluids. Non-limiting examples of such derivatives include:

Cysteinyl residues which are most commonly reacted with alpha-haloacetates (and corresponding amines), such as chloroacetic acid or chloroacetamide, to give carboxymethyl or carboxyamidomethyl derivatives. Cysteinyl residues also are derivatized by reaction with bromotrifluoroacetone, alphabromo-beta-(5-imidazoyl) propionic acid, chloroacetyl phosphate, N-alkylmaleimides, 3-nitro-2-pyridyl disulfide, methyl-2pyridyl disulfide, p-chloromercuribenzoate, 2-chloromercuri-4-nitrophenol, or chloro-7-nitrobenzo-2-oxa-1,3-diazole.

Histidyl residues are derivatized by reaction with diethylprocarbonate. at pH 5.5-7.0 because this agent is relatively specific for the histidyl side chain. Parabromophenacyl bromide also is useful; the reaction is preferably performed in 0.1 M sodium cacodylate at pH 6.0.

Lysinyl and amino terminal residues are reacted with succinic or other carboxylic acid anhydrides. Derivatization with these agents has the effect of reversing the charge of the lysinyl residues. Other suitable reagents for derivatizing alpha-amino-containing residues include imidoesters such as methyl picolinimidate; pyridoxal phosphate; pyridoxal; chloroborohydride; trinitrobenzenesulfonic acid; O-methylisourea; 2,4-pentanedione; and transaminase-catalyzed reaction with glyoxylate.

Arginyl residues are modified by reaction with one or several conventional reagents, among them phenylglyoxal, 2,3 butanedione, 1,2-cyclodexanedione, and ninhydrin.

Derivatization of arginine residues requires that the reaction be performed in alkaline conditions because of the high pKa of the guanidine functional group. Furthermore, these reagents may react with the groups of lysine as well as the arginine epsilon-amino group.

The specific modification of tyrosyl residues per se has been studied extensively, with particular interest in introducing spectral labels into tyrosyl residues by reaction with aromatic diazonium compounds or tetranitromethane. Most commonly, N-acetylimidazole and tetranitromethane are used to form O-acetyl tyrosyl species and 3-nitro derivatives, respectively.

Carboxyl side groups (aspartyl or glutamyl) are selectively modified by reaction with carbodiimides (R′—N—C—N—R′), such as 1-cyclohexyl-3-[2-morpholinyl-(4-ethyl)]carbodiimide or 1-ethyl-3-(4-azonia-4,4-dimethlypentyl) carbodiimide. Furthermore, aspartyl and glutamyl residues are converted to asparaginyl and glutaminyl residues by reaction with ammonium ions.

Glutaminyl and asparaginyl residues are frequently deamidated to the corresponding glutamyl and aspartyl residues. Alternatively, these residues are deamidated under mild acidic conditions. Either form of these residues falls within the scope of this invention.

The term “derivatives” is intended to include only those derivatives that do not change one amino acid to another of the twenty commonly-occurring natural amino acids.

The term “salts” herein refers to both salts of carboxyl groups and to acid addition salts of amino groups of the complex of the invention or analogs thereof. Salts of a carboxyl group may be formed by means known in the art and include inorganic salts, for example, sodium, calcium, ammonium, ferric or zinc salts, and the like, and salts with organic bases as those formed, for example, with amines, such as triethanolamine, arginine or lysine, piperidine, procaine and the like. Acid addition salts include, for example, salts with mineral acids, such as, for example, hydrochloric acid or sulfuric acid, and salts with organic acids, such as, for example, acetic acid or oxalic acid. Of course, any such salts must have substantially similar biological activity to the complex of the invention or its analogs.

The term “fragment” of the enzyme alliinase or a variant thereof is intended to cover any fragment of alliinase or an analog thereof that retains the catalytic activity and ability to generate allicin in a lyase reaction from alliin substrates. For example, fragments can be readily generated from alliinase where successive residues can be removed from either the N-terminus or C-terminus or both of alliinase, or from peptides obtained therefrom by enzymatic or chemical cleavage of the polypeptide. Thus, multiple substitutions are not involved in screening for catalytically active fragments of alliinase. If the removal of one or two amino acids from one end or the other does not affect the catalytic activity after testing in the standard tests, as discussed herein, such truncated polypeptides are considered to be within the scope of the present invention. Further truncations can then be carried out until it is found where the removal of another residue destroys the catalytic activity. Alliinase can be attached to the monoclonal antibodies of the invention by chemical means by generating covalently bound conjugates, as well as by physical means such as by affinity binding, entrapping the enzyme within a polymer matrix or membrane (liposome), or microencapsulating the enzyme within semipermeable polymer membranes. According to currently preferred embodiments, the antibody of the invention is chemically ligated to the enzyme alliinase.

Any suitable technique used to prepare conjugates for biotechnological or medical applications can be used for the present invention. One example is the technique described in Miron et al., International Publication WO 97/39115. Other chemical conjugation methods are described in Epstein et al, U.S. Pat. No. 6,008,319 (note particularly col. 7, lines 2260). The entire contents of both of these patents are incorporated herein by reference. The alliinase can be bound to the antibody directly or through a spacer.

Functional groups of the antibodies such as thiol, hydroxyl, amino, and carboxyl groups may be activated and thus used for coupling with alliinase. Coupling can be affected by various chemical crosslinking methods, forming peptide (amide), azo, thioether, disulfide, and other bonds. Any of these methods can be used for this invention. For example, to form a peptide bond, carboxyl groups of the antibody are converted to reactive derivatives such as N-hydroxysuccinimide ester, and these derivatives form peptide bonds with free amino groups of alliinase. It is also possible to form peptide bonds between free carboxyl or amino groups of the enzyme, and amino groups or carboxyl groups of the antibody, respectively, using condensing agents such as cabodiimides and Woodward's reagent K. Another possibility is to use a molecule which serves as a spacer, e.g., epsilon-aminocaproic acid, 3-(2-pyridyldithio) propionic acid N-hydroxysuccinimide ester (SPDP) or succinimidyl-[(N-maleimidopropionamido)-tetraethyleneglycol]ester) (NHS-PEO₄-Maleimide). The spacer is first attached either to the antibody or to alliinase through an amino group. Then, using the methods described above, the two entities are coupled. According to some embodiments, alliinase may be coupled with polyethylene glycol (PEG) prior to being coupled to the antibody. The PEG linker may have a molecular weight of 100 to 5000 kD, preferably 100 to 500 kD. The pegylation of alliinase improves the water solubility of the conjugate, reduces the potential for conjugate aggregation and increases the flexibility of the crosslink between the alliinase and the antibody, thus stabilizing the conjugate.

For stabilization or for immunological reasons, alliinase can be coupled with various biocompatible synthetic polymers such as methoxypolyethylene glycol (mPEG) prior to being coupled to the antibody. Pegylation of alliinase is carried out by standard techniques, well known to one skilled in the art.

Alliinase can be coupled with the antibody using, for example, biotinylated alliinase or haptenized alliinase that can interact with avidin on the antibody.

Another approach for preparing conjugates of the present invention consists in developing recombinant antibody-alliinase fusion proteins (also referred to as “recombinant conjugate”), consisting of single molecular entities. Genetically engineered fusion proteins may be constructed by cloning the gene sequences of antibody light chains and heavy chains fused to sequences encoding alliinase. As an example, mRNA from hybridoma cells expressing a monoclonal antibody is isolated. From this mRNA, cDNA is reverse transcribed and amplified by polymerase chain reaction. Specific regions encoding heavy and light chains of an immunoglobulin, e.g., variable and/or constant regions, can be amplified by the selection of appropriate oligonucleotide primers targeting the desired region(s). The cDNA is sequenced, mapped by restriction endonucleases, and cloned into an appropriate transfer vector. At a minimum, the immunoglobulin sequences encoding an antigen binding domain, i.e., the variable light chain and variable heavy chain regions, are contained in the transfer vector. In addition, a truncated or full-length portion of the constant region encoding the original or another immunoglobulin can be joined in frame with the variable region, to allow expression of the joined regions. For example, a preferred embodiment of the invention encodes a chimeric mAb, comprised of murine variable regions linked to their corresponding human constant regions of the heavy and light chains. An appropriate DNA sequence, encoding at least one alliinase peptide, is then ligated proximate to a region of an immunoglobulin gene encoding the carboxy-terminus, preferably a constant region, most preferably the constant region of a heavy chain. The best site for attachment for each alliinase may be different and may be easily determined via experimental methods. For example, none or various lengths of amino acid encoding linkers may be inserted between the alliinase and the carboxy-terminus of the immunoglobulin gene. The resulting expression products can then be tested for biologic activity.

The completed engineered gene for the fusion protein is inserted into an expression vector, which can be introduced into eukaryotic or prokaryotic cells by gene transfection methods, e.g., electroporation or the calcium phosphate method. The fusion protein product can then be expressed in large-scale cell culture and purified.

As used herein, the term “vector” refers any recombinant polynucleotide construct that may be used for the purpose of transformation, i.e. the introduction of heterologous DNA into a host cell. One type of vector is a “plasmid”, which refers to a circular double stranded DNA loop into which additional DNA segments can be ligated. Another type of vector is a viral vector, wherein additional DNA segments can be ligated into the viral genome. Yet another type of vector is a plant vector (e.g. plant binary vector pE1801). Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”.

An “expression vector” as used herein refers to a nucleic acid molecule capable of replication and expressing a gene of interest when transformed, transfected or transduced into a host cell. The expression vectors comprise one or more phenotypic selectable markers and an origin of replication to ensure maintenance of the vector and to, if desired, provide amplification within the host. Selectable markers include, for example, sequences conferring antibiotic resistance markers, which may be used to obtain successful transformants by selection, such as ampicillin, tetracycline and kanamycin resistance sequences, or supply critical nutrients not available from complex media. The expression vector further comprises a promoter. In the context of the present invention, the promoter must be able to drive the expression of the polypeptide within the cells. Suitable eukaryotic promoters include promoters which contain enhancer sequences (e.g., the rabbit β-globin regulatory elements), constitutively active promoters (e.g., the β-actin promoter, etc.) and signal and/or tissue specific promoters (e.g., inducible and/or repressible promoters, such as a promoter responsive to TNF or RU486, the metallothionine promoter, etc.). Suitable expression vectors may be plasmids derived, for example, from pBR322 or various pUC plasmids, which are commercially available.

Other expression vectors may be derived from bacteriophage, phagemid, or cosmid expression vectors, all of which are described in sections 1.12-1.20 of Sambrook et al., (Molecular Cloning: A Laboratory Manual. 3^{rd} edn., 2001, Cold Spring Harbor Laboratory Press). Isolated plasmids and DNA fragments are cleaved, tailored, and ligated together in a specific order to generate the desired vectors, as is well known in the art (see, for example, Sambrook et al., ibid).

Methods for manipulating a vector comprising an isolated polynucleotide are well known in the art (e.g., Sambrook et al., 1989, Molecular Cloning: A Laboratory Manual, 2d edition, Cold Spring Harbor Press, the contents of which are hereby incorporated by reference in their entirety) and include direct cloning, site specific recombination using recombinases, homologous recombination, and other suitable methods of constructing a recombinant vector. In this manner, an expression vector can be constructed such that it can be replicated in any desired cell, expressed in any desired cell, and can even become integrated into the genome of any desired cell.

The expression vector comprising the polynucleotide of interest is introduced into the cells by any means appropriate for the transfer of DNA into cells. Many such methods are well known in the art (e.g., Sambrook et al., supra; see also Watson et al., 1992, Recombinant DNA, Chapter 12, 2d edition, Scientific American Books, the contents of which are hereby incorporated by reference in their entirety). Thus, in the case of prokaryotic cells, vector introduction can be accomplished, for example, by electroporation, transformation, transduction, conjugation, or mobilization. For eukaryotic cells, vectors can be introduced through the use of, for example, electroporation, transfection, infection, DNA coated microprojectiles, or protoplast fusion. Examples of eukaryotic cells into which the expression vector can be introduced include, but are not limited to, ovum, stem cells, blastocytes, and the like. According to some embodiments of the present invention, the C-terminal end of alliinase, or any biologically active analog or fragment thereof, may be fused to the N-terminal end of an immunoglobulin chain, preferably a single chain antibody (scFv) or an antigen-binding fragment thereof. The reverse constructs can also be prepared, where the C-terminal end of the antibody chain is fused to the N-terminal end of the alliinase molecule. In order to engineer an alliinase/antibody fusion protein that allows the active complex to be maintained, a flexible peptide linker, for example Gly-Gly-Gly-Gly-Ser (GGGGS) repeats, may be employed. Preferably these linkers are up to about 30 amino acids in length. Such recombinant conjugates have the advantage of being highly reproducible with respect to both their production and 3D structure. In addition, such recombinant conjugate can be easily purified in large quantities.

The present invention also concerns DNA sequences encoding the above fusion protein of the invention, as well as DNA vectors carrying such DNA sequences for expression in suitable prokaryotic or eukaryotic host cells. The ability to generate large quantities of heterologous proteins using a recombinant protein expression system has led to the development of various therapeutic agents. The various expression hosts from which recombinant proteins can be generated range from prokaryotic in origin (e.g., bacteria), through lower eukaryotes (e.g., yeast), plants (e.g., tomato, tobacco (for example: Pogrebnyak N. et al., (2005), Proc. Nat. Acad. Sci., 102:9062-7 and cited references therein)), to higher eukaryotic species (e.g., insect and mammalian cells). All of these systems rely upon the same principle, i.e., introducing the DNA sequence of the protein of interest into the chosen cell type (in a transient or stable fashion, as an integrated or episomal element) and using the host transcription, translation and transportation machinery to over-express the introduced DNA sequence as a heterologous protein.

Other techniques for making fusion proteins between alliinase and the antibody of the invention or fragments thereof are disclosed in the patents listed and incorporated by reference hereinabove.

### Pharmaceutical Composition

The present invention provides a pharmaceutical composition comprising a monoclonal antibody according to embodiments of the invention and a pharmaceutically acceptable carrier.

The present invention further provides a pharmaceutical composition comprising a conjugate of the invention and a pharmaceutically acceptable carrier.

While any suitable formulation of the compositions is encompassed by the invention, preferably, it will be adapted for inhalation, intratracheal instillation oral and/or intranasal administration.

Pharmaceutical compositions according to the present invention can be administered by any convenient route, including oral, intratracheal, intranasal, parenteral, subcutaneous, intravenous, intramuscular, intraperitoneal, transdermal or topical. The dosage administered depends upon the age, health, and weight of the recipient, nature of concurrent treatment, if any, and the nature of the effect desired.

Compositions within the scope of the present invention include all compositions wherein the conjugate is contained in an amount effective to achieve its intended purpose. While individual needs vary, determination of optimal ranges of effective amounts of each compound is within the skill of the art. As the conjugate is non-toxic and, unless it binds to its target cell, is removed quickly from the system, there is, practically, no maximum dosage amount. Typical preferred dosages comprise 0.01 to 100 mg/kg body weight.

Pharmaceutical compositions for administering the active ingredients of the present invention preferably contain, in addition to the pharmacologically active compound, suitable pharmaceutically acceptable carriers comprising excipients and auxiliaries which facilitate processing of the active compounds into preparations which can be used pharmaceutically.

Preferably, the preparations contain from about 0.01 to about 99 percent by weight, preferably from about 20 to 75 percent by weight, active compound (s), together with the excipient. For purposes of the present invention, all percentages are by weight unless otherwise indicated.

The pharmaceutically acceptable carriers include vehicles, adjuvants, excipients, or diluents that are well known to those skilled in the art and which are readily available. It is preferred that the pharmaceutically acceptable carrier be one which is chemically inert to the active compounds and which has no detrimental side effects or toxicity under the conditions of use.

The choice of carrier is determined partly by the particular active ingredient, as well as by the particular method used to administer the composition. Accordingly, there is a wide variety of suitable formulations of the pharmaceutical compositions of the present invention. While the preferred route for administering the conjugates of the present invention is via inhalation or intratracheal instillation, formulations can also be prepared for oral, aerosol, parenteral, subcutaneous, intravenous, intraarterial, intramuscular, intraperitoneal, rectal, and vaginal administration.

Suitable formulation for administration via inhalation may include an aerosol spray from pressurized packs or a nebulizer with the use of a suitable propellant, e.g., dichlorodifluoromethane, trichlorofluoromethane, carbon dioxide or other suitable gas. In the case of a pressurized aerosol, the dosage unit may be determined by providing a valve to deliver a metered amount. Capsules and cartridges of, e.g., gelatin, for use in an inhaler or insufflator may be formulated containing a powder mix of the conjugate or alliin and a suitable powder base such as lactose or starch.

Suitable formulations for parenteral administration include aqueous solutions of the active compounds in water soluble form, such as water-soluble salts. In addition, suspensions of the active compounds as appropriate oily injection suspensions may be administered. Suitable lipophilic solvents or vehicles include fatty oils, for example, sesame oil, or synthetic fatty acid esters, for example, ethyl oleate or triglycerides. Aqueous injection suspensions may contain substances which increase the viscosity of the suspension, including, for example, sodium carboxymethyl cellulose, sorbitol, and/or dextran. Optionally, the suspension may also contain stabilizers.

Other pharmaceutically acceptable carriers for the active ingredients according to the present invention are liposomes, pharmaceutical compositions in which the active ingredient is contained either dispersed or variously present in corpuscles contained either dispersed or variously present in corpuscles consisting of aqueous concentric layers adherent to lipid layers. The active ingredient may be present both in the aqueous layer and in the lipidic layer, inside or outside, or, in any event, in the non-homogeneous system generally known as a liposomic suspension.

The hydrophobic layer, or lipid layer, generally, but not exclusively, comprises phospholipids such as lecithin and sphingomyelin, steroids such as cholesterol, more or less ionic surface active substances such as dicetyl phosphate, stearylamine, or phosphatidic acid, and/or other materials of a hydrophobic nature.

Liquid formulations may include diluents such as water and alcohols, e.g., ethanol, benzyl alcohol, and the polyethylene alcohols, either with or without the addition of a pharmaceutically acceptable surfactant, suspending agents, or emulsifying agents. Capsule forms can be of the ordinary hard or soft-shelled gelatin type containing, for example, surfactants, lubricant, and inert fillers, such as lactose, sucrose, calcium phosphate, and corn starch. Tablet forms can include one or more of lactose, sucrose, mannitol, corn starch, potato starch, alginic acid, microcrystalline cellulose, acacia, gelatin, guar gum, colloidal silicon dioxide, croscaramellose sodium, talc, magnesium stearate, calcium stearate, zinc stearate, stearic acid, and other preservatives, flavoring agents, and pharmaceutically acceptable disintegrating agents, moistening agents preservatives flavoring agents, and pharmacologically compatible carriers.

Lozenge forms can comprise the active ingredient in a carrier, usually sucrose and acacia or tragacanth, as well as pastilles comprising the active ingredient in an inert base such as gelatin or glycerin, or sucrose and acacia. Emulsions and the like can contain, in addition to the active ingredient, such carriers as are known in the art.

Formulations suitable for parenteral administration include aqueous and non-aqueous, isotonic sterile injection solutions, which can contain anti-oxidants, buffers, bacteriostatic agents, and solutes that render the formulation isotonic with the blood of the intended recipient, and aqueous and nonaqueous sterile suspensions that can include suspending agents, solubilizers, thickening agents, stabilizers, and preservatives. The compounds can be administered in a physiologically acceptable diluent in a pharmaceutical carrier, such as a sterile liquid or mixture of liquids, including water, saline, aqueous dextrose and related sugar solutions, an alcohol such as ethanol, isopropanol, or hexadecyl alcohol, glycols such as propylene glycol or polyethylene glycol, glycerol ketals such as 2,2-dimethyl-1,3-dioxolane-4-methanol, ethers such as poly (ethylene glycol) 400, oils, fatty acids, fatty acid esters or glycerides, or acetylated fatty acid glycerides, with or without the addition of a pharmaceutically acceptable surfactant, such as soap or a detergent, suspending agent, such as carbomers, methylcellulose, hydroxypropylmethylcellulose, or carboxymethylcellulose, or emulsifying agents and other pharmaceutical adjuvants.

Oils which can be used in parenteral formulations include petroleum, animal, vegetable, or synthetic oils. Specific examples of oils include peanut, soybean, sesame, cottonseed, corn, olive, petrolatum, and mineral. Fatty acids can be used in parenteral formulations, including oleic acid, stearic acid, and isostearic acid. Ethyl oleate and isopropyl myristate are examples of suitable fatty acid esters. Suitable salts for use in parenteral formulations include fatty alkali metal, ammonium, and triethanolamine salts, and suitable detergents include cationic detergents such as dimethyl dialkyl ammonium halides, and alkyl pyridinium halides; anionic detergents such as dimethyl olefin sulfonates, alkyl, olefin, ether, and monoglyceride sulfates and sulfosuccinates; polyoxyethylenepolypropylene copolymers; amphoteric detergents such as alkyl-ss-aminopropionates and 2-alkyl-imidazoline quaternary ammonium salts; and mixtures thereof.

Parenteral formulations typically contain from about 0.5 to 25% by weight of the conjugate in solution or suspension. Suitable preservatives and buffers can be used in these formulations. In order to minimize of eliminate irritation at the site of injection, these compositions may contain one or more nonionic surfactants having a hydrophilic lipophilic balance (HLB) of from about 12 to about 17. The quantity of surfactant in such formulations ranges from about 5 to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol. The parenteral formulations can be present in unit dose or multiple dose sealed containers, such as ampoules and vials, and can be stored in a freeze-dried (lyophilized) condition requiring only the addition of the sterile liquid carrier, e.g., water, for injections immediately prior to use. Extemporaneous injection solutions and suspensions can be prepared from sterile powders, granules, and tablets of the kind previously described.

In determining the dosages of the conjugate and alliin to be administered, the dosage and frequency of administration is selected in relation to the pharmacological properties of the specific active ingredients. Normally, at least three dosage levels should be used. In toxicity studies in general, the highest dose should reach a toxic level but be sublethal for most animals in the group. If possible, the lowest dose should induce a biologically demonstrable effect.

The amount of conjugate of the present invention and of alliin to be administered to any given patient must be determined empirically, and will differ depending upon the condition of the patients. Relatively small amounts of the active ingredients can be administered at first, with steadily increasing dosages if no adverse effects are noted. Of course, the maximum safe toxicity dosage as determined should not be exceeded.

The present invention also provides a method for treating diseases or conditions caused by Aspergillus that the method comprises administering to an individual in need thereof a conjugate of the enzyme alliinase with the monoclonal antibody of the invention or fragments thereof, that targets the conjugate to an Aspergillus species found in the body, followed by administration of alliin. In this way, allicin is generated at the desired Aspergillus species, thus exerting its biological activity. It is to be emphasizes, that the disease or disorder caused by Aspergillus according to embodiments of the present invention is treatable with allicin.

In treating a disorder or disease treatable with allicin, the conjugate is administered first, followed by administration of alliin. Preferably, the alliin is administered from about 30 minutes to up about five hours after administration of the conjugate. The administration of alliin may be repeated one or several times as necessary, in intervals to be decided according to the stage of the disease and condition of the patient.

Administration of alliin, which is a non-toxic amino acid derivative, essentially potentiates the monoclonal antibody-alliinase conjugate located on an Aspergillus cell. The enzymatic activity of alliinase in the conjugate enables continuous generation of allicin at the Aspergillus cell site. Generation of allicin depends on the availability of the substrate alliin. Since mammalian cells do not produce a lyase type of enzyme such as alliinase, the administration of alliin poses no toxic danger, as the alliin is converted to allicin only by the alliinase which is ligated to the monoclonal antibody or fragments thereof.

Since Aspergillus cells treated only with alliin (in the absence of the conjugate) or only with the conjugate (without the addition of alliin) were not inhibited in their growth, the conjugates of the present invention clearly have a very wide range of potential applications.

### EXPERIMENTAL DETAILS SECTION

### Materials and Methods

Fungal strains Aspergillus fumigatus strain 293 and the clinical isolate CBS 144.89 (provided by the laboratory of Prof. Jean-Paul Latgé, Aspergillus Unit, Pasteur Institute, Paris) were used for in vitro experiments. The fluorescent strain (CBS 144.89/DsRed), previously described (Vallon-Eberhard et al., 2008, Antimicrob. Agents Chemother. 52:3118-26), was used as an infection read-out in mice. Resting conidia were counted with a hemacytometer and grown in RPMI-MOPS (Shadkchan et al., 2004, J. Antimicrob. Chemother., 53:832-6). Other fungal strains tested for the binding of the anti Aspergillus fumigatus (AF) mAb MPS 5.44 were Aspergillus niger, Aspergillus flavus and Aspergillus terreus, Candida albicans and Candida krusei.

### Preparation of Pure Allicin:

Pure allicin was produced by passing a solution of synthetic, nature-identical alliin (see below), through an immobilized alliinase column (Miron et al., 2000, Biochim. Biophys. Acta, 1463:20-30). Allicin was analyzed and quantified by HPLC as described by Miron et al., 1998, Anal. Biochem., 265:317-25.

### Preparation of a mAb:

Anti-AF mAbs were produced in mice. Balb/c mice (Harlan, Jerusalem) were injected subcutaneously with preparation containing freshly harvested AF293 conidia and hyphae at 2 weekly intervals, first with complete Freund's adjuvant and on the second and third boosts with incomplete Freund's adjuvant. The fourth dose was given intraperitoneally, without adjuvant. Three mice used for fusion were boosted with the antigen (freshly harvested AF293 conidia and hyphae) 1 month after the fourth dose and the spleen was removed 4 days later. Splenocytes were obtained from the immunized mice and fused with NSO myeloma cells at ratio of 10:1, with 50% polyethylene glycol 1500 (0.8 ml 50% PEG per 1×10⁸ spleen cells+1×10⁷ NSO). The hybrids were plated out into 96-well plates in DMEM buffer containing 20% horse serum and 2× oxaloacetate/pyruvate/insulin (Sigma), and hypoxanthine/aminopterin/thymidine selection was begun. On day 8, 100 μl of DMEM buffer containing 20% horse serum was added to all the wells. Supernatants of the hybrids were screened for binding to Aspergillus Fumigatus (AF) 293 hyphae. Positive hybridomas were cloned twice by limiting dilution. Determination of the antibody class was done with class-specific second antibody. Clone MPS5.44 class IgM displayed the highest binding affinity and was chosen for further work. Large quantities of specific antibodies were produced from the hybridoma cell culture medium and were purified by affinity chromatography on MBP-columns (Pierce).

Anti-dinitrophenol IgM mAb purified as described for MPS 5.44 was used as a control._These antibodies were obtained from the respective hybridoma following an identical procedure to the procedure described above.

The Amino acid sequences of VH (heavy chain variable region) and VL (light chain variable region) of the mAbs were determined using Mass spectrometry. The N-terminus amino acid sequence of the heavy chain was revealed by Edman degradation.

For sequencing the DNA of the VH and VL variable regions of the mAbs, mRNA was isolated from approximately 1×10⁷ hybridoma cells, using TriReagent (Sigma), according to the manufacturer's instructions and reverse-transcribed (1 μg RNA) by AMV reverse transcriptase (Promega) and oligo-dT as a primer. VL was amplified by PCR with the degenerative primer Ksignal forward 5′-ATGGAYTTYCARGTNCARATHTT-3′ (SEQ ID NO:10) and primer Kas reverse 5′-YTTYTGYTGRTACCARTGCAT-3′ (SEQ ID NO:11) primers, whereas VH was amplified with the LVQP forward 5′-CTGGTGCAGCCTGGAGGATCCC-3′ (SEQ ID NO:12) and ALYY reverse 5′-TCTTGCACAGTAATAAAGGGC-3′ (SEQ ID NO:13) primers. The products of the PCR reactions were analyzed by sequencing with the same primers. VH and VL CDRs were analyzed with the Blast/immunoglobulin database.

Preparation of the mAb-Alliinase Conjugates:

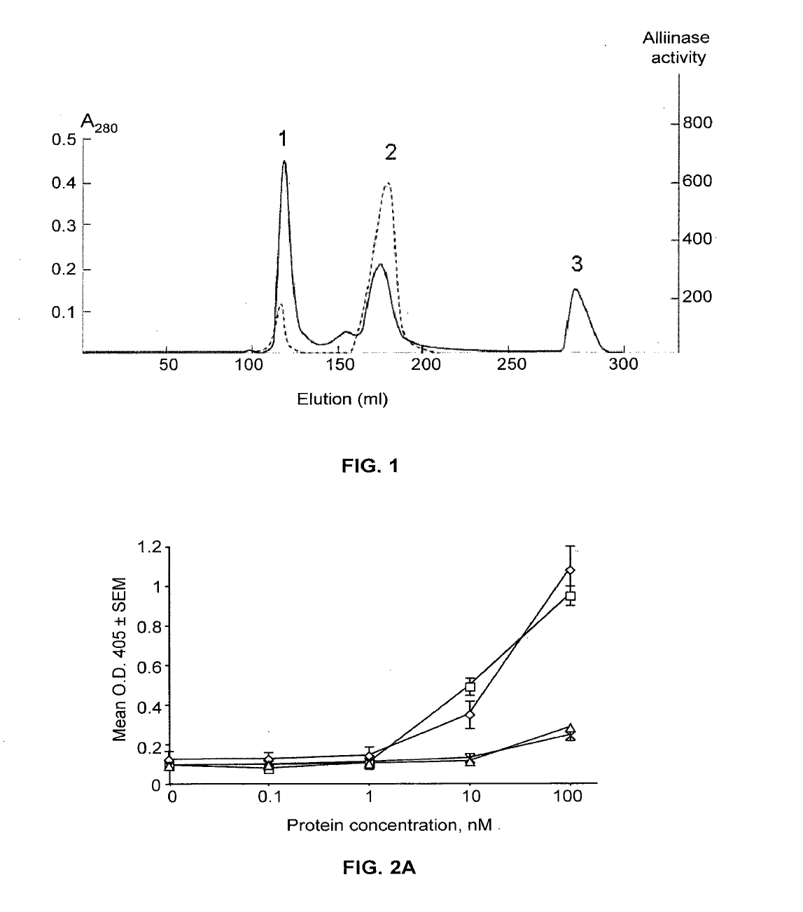

Alliinase was purified from garlic cloves as previously described (Rabinkov et al., 1995, Glycoconj. J, 12:690-8). Conjugation of mAbs with alliinase was performed in three steps: (i) thiolation of the mAbs with iminothiolane according to Lambert et al., 1985, J. Biol. Chem., 260:12035-41; (ii) derivatization of alliinase with NHS-PEO₄-maleimide and (iii) conjugation between the two modified proteins according to the manufacturer's protocol (Pierce Co.). The molar ratio of mAb:alliinase taken for conjugation was 1:3. The high molecular weight conjugates (MW˜1,200 kDa) were separated from free alliinase (MW 100 kDa) by size exclusion chromatography (FIG. 1). Alliinase activity of the fractions was determined using the NTB method as described by Miron et al., 1998, Anal. Biochem., 265:317-25. Synthetic, nature-identical alliin (α=+63°) was used as the substrate and was synthesized from L-cysteine and allyl-bromide followed by H₂O₂ oxidation as described by Stoll and Seebeck, 1951, Helv. Chim. Acta., 34:481-7. A unit (U) of alliinase activity is defined as the amount of enzyme required to release 1 μmol of pyruvate per min (Miron et al., 1998, Anal. Biochem., 265:317-25). The chemical conjugation did not impair alliinase activity. The specific activity of free alliinase and of the alliinase in the mAb-alliinase conjugates was found to be 100 U/nmol and 125 U/nmol respectively. The dominant population of the conjugate molecules was determined by dynamic light scattering and showed a mean hydrodynamic size of approximately 100 nm. Assuming a spherical shape for the conjugate and an average density of 1.2 g/cc, an estimated diameter of the conjugate is of about of 120 nm.

Recombinant Alliinase: The synthesis and cloning of a cDNA Fragment Coding for Alliinase by MOPAC (Mixed Oligonucleotide Primed Amplification of cDNA), construction of alliinase cDNA libraries was performed as fully described in Rabinkov A. et al., Appl. Biochem. Biotechnol. (1994) 48:149-71.
Sequences of the Anti-Aspergillus Monoclonal Antibody (from MPS 5.44 Hybridoma):

The mAb antibodies were purified and sequences of variable chains and CDRs were identified:
CDR_{H1}—RFWMS (SEQ ID NO:1);
CDR_{H2}—EINPESSTINYTPSLKD (SEQ ID NO:2);
CDR_{H3}—DAGPYGNYFDY (SEQ ID NO:14);
CDR_{L1}—SASSSVRYMH (SEQ ID NO:3);
CDR_{L2}—DTSEKAS (SEQ ID NO:4);
CDR_{L3}—QQWSSHPFT (SEQ ID NO:5);

FWR_{L1}-CDR_{L1}-FWR_{L2}-CHR₂-FWR₃
QIVLTQSPAIMSASPGEKVTMTCSASSSVRYMHWYQQKSGTSPKRWIY DTSEKASGVPTRFSGSGSGTSYSLTISTMEAEDAATYYC (SEQ ID NO:6);

FWR_{H1}-CDR_{H1}-FWR_{H2}-CHR_{H2}-FWR_{H3}
EVKLLESGGGLVQPGGSLKLSCAASGFDFSRFWMSWVRQAPGKGLEWI GEINPESSTINYTPSLKDKFIISRDNAKNTLYLQMSKVRSEDTALYYCAR (SEQ ID NO:7);

Sequence of Kappa Light Chain of MPS5.44 Reconstructed by Sequencing with Oligos Kas and Ksignal:
atggattucaggtgcagatutcagcttcctgctaatcagtgcctcagtcataatatccagaggacaaattgttctcac ccagtctccagcaatcatgtctgcatctccaggggagaaggtcaccatgacctgcagtgccagctcaagtgtacgt tacatgcactggtaccagcagaagtcaggcacctcccccaaaagatggatttatgacacatccgaaaaggcttctg gagtccctactcgcttcagtggcagtgggtctgggacctcttactactcacaatcagcaccatggaggctgaagat gctgccacttattactgccagcagtggagtagtcacccattcacgttcggctcggggacaaaattggaaataaaac gggctgatgctgcaccaactgtatccatcttcccaccatccagtgagcagttaacatctggaggtgcctcagtcgtg tgcttcttgaacaacttctaccccaaagacatcaatgtcaagtggaagattgatggcagtgaacgacaaaatggcgt cctgaacagttggactgatcaggacagcaaagacagcacctacagcatgagcagcaccctcacgttgaccaagg acgagtatgaacgacataacagctatacctgtgaggccactcacaagacatcaacttcacccattgtcaagagcttc aacaggaatgagtgttag (SEQ ID NO:8);

Fv of Heavy Chain Reconstructed by Sequencing with Oligos LVQP and ALYY and Based on Edman Degradation:
gaggtgaagcttacgagtctggaggtggcctggtgcagcctggaggatccctgaaactctcctgtgcagcctca ggattcgattttagtagattctggatgagagggtccggcaggctccagggaaagggctagaatggattggagaaa ttaatccagagagcagtacgataaactatacgccatactaaaggataaattcataataccagagacaacgccaaa aatacgctgtacctgcaaatgagcaaagtgagatctgaggacacagccctttattactgtgcaaga (SEQ ID NO:9).

Mouse Heavy Chain Fv Amino Acid Sequence from Mass Spectra:

EVKLLESGGGLVQPGGSLKLSCAASGFDFSRFWMSWVRQAPGKGLEWI GEINPESSTINYTPSLKDKFIISRDNAKNTLYLQMSKVRSEDTALYYCAR DAGPYGNYFDYWGQGTTLTVS SESQSFPNVFPLVSCESPLSDKNLVAM GCLARDFLPSTISFTWNYQNNTEVIQGIRTFPTLRTGGKYLATSQVLLSP KSILEGSDEYLVCKIHYGGKNRDLHVPIPAVAEMNPNVNVFVPPRDGFS GPAPRKSKLICEATNFTPKPITVSWLICDGKLVESGFTTDPVTIENKGSTP QTYKVISTLTISEIDWLNLNVYTCRVDHRGLTFLKNVSSTCAASPSTDIL TFTIPPSFADIFLSKSANLTCLVSNLATYETLNISWASQSGEPLETKIKIME SHPNGTFSAKGVASVCVEDWNNRKEFVCTVTHRDLPSPQKKFISKPNEV HKHPPAVYLLPPAREQLNLRESATVTCLVKGFSPADISVQWLQRGQLLP QEKYVTSAPMPEPGAPGFYFTHSILTVTEEEWNSGETYTCVVGHEALPH LVTERTVDKSTGK (SEQ ID NO:15).

The gene coding for the scFv chain (SEQ ID NOS: 8 and 9) was amplified by RT PCR from RNA extracted from the hybridoma cell line that produced the mAb. The forward and reverse PCR primers used were designed from the Edman degradation sequences and then from the nucleotide sequence of gene of the anti-Aspergillus scFv chain. The gene coding for the scFv chain is preferably expressed using mammalian expression vectors (e.g. pCMX2.5-hIgG1-Fc-XP) or any other available vectors which enable the fusion of scFv antibodies to the gene fragment encoding the human IgG1 Fc part (hinge-CH₂—CH₃) and allows the production of homodimeric scFv-Fc fusion proteins in HEK293 T cells.

### Example 1

### Binding Properties of the mAb (MPS 5.44)-Alliinase Conjugate to Aspergillus fumigatus (ELISA)

The binding of the mAb-alliinase conjugate or the unconjugated MPS 5.44 antibodies to either Aspergillus fumigatus (AF) hyphae or swollen conidia was performed in 96 well plates in triplicates. A non-specific IgM antibody (anti-DNP) served as a negative control. Blocking of non-specific binding was done by pre-incubation (37° C., 1 hour) of the conidia or hyphae with a solution of haemoglobin (1%) in TBST buffer (20 mM Tris pH 8.0, 140 mM NaCl and 0.05% Tween-80). Plates were then washed twice with TBST buffer and incubated with serial dilutions of the above antibodies in TBS buffer (20 mM Tris pH 8.0 and 140 mM NaCl) for 30 min. Unbound antibodies were removed by four consecutive washings with TBST buffer and incubated for 1 hour with a secondary goat anti-mouse μ-chain antibody conjugated to alkaline phosphatase (Sigma, Cat # A7784), diluted 1:10,000 in 1% hemoglobin/TBST buffer. Plates were washed with TBST buffer four times and then incubated with the alkaline phosphatase substrate, p-Nitrophenyl Phosphate (PNPP) (1 mg/ml in buffer 0.2M Tris pH 9.5, 100 mM NaCl and 5 mM MgCl₂), at RT for 1 hour. Color intensity was determined with an ELISA reader at OD₄₀₅.

### Results:

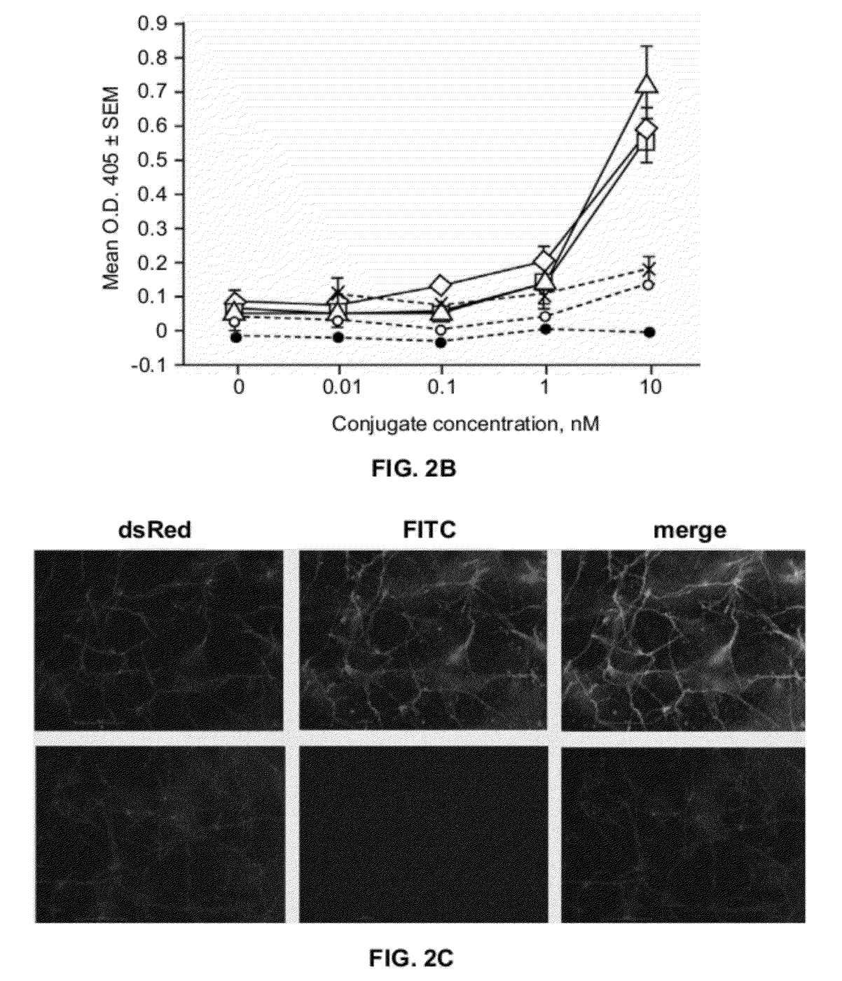

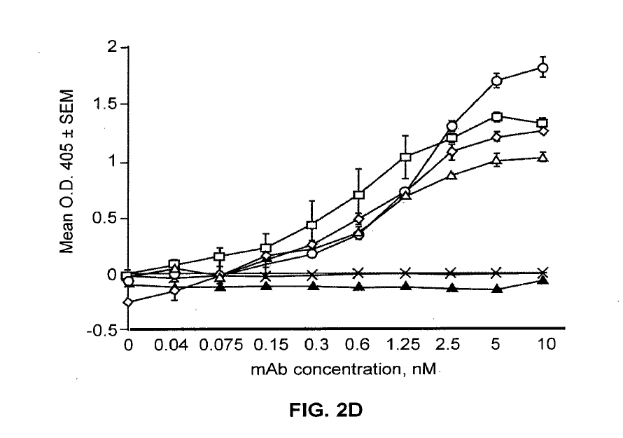

The binding of the Aspergillus cell wall-specific non-conjugated mAb (MPS5.44) was compared to that of the mAb(MPS5.44)-alliinase conjugates. It was found that both proteins bound Aspergillus fumigatus (AF) hyphae and swollen conidia at a similar concentrations (1-10 nM, FIG. 2A). Curve fit analysis indicated no statistically significant difference between the binding curves of the mAb(MPS5.44)-alliinase conjugate and the free mAb (p>0.05). Notably, at these concentrations, no significant binding was detected with the non-specific IgM antibody used as a control or with the conjugate prepared with the non-specific mAb (FIG. 2A). Conjugates of mAb (MPS5.44)-alliinase bound all AF forms, i.e. resting or swollen conidia as well as hyphae at similar concentrations (FIG. 2B). A comparison between the binding of an FITC-labeled conjugate with that of FITC-labeled free alliinase to the dsRed-AF hyphae was made using fluorescent microscopy (FIG. 2C). The results show that the binding of the conjugate to the red fluorescent hyphae (dsRed) was visible (FIG. 2C, top panel) whereas binding of free, non conjugated alliinase (FIG. 2C, lower panel) was almost undetectable. This demonstrates that the non-specific adherence of the unconjugated alliinase to hyphae, is much weaker than the binding of mAb MPS 5.44 and that the anchoring of the conjugate is due to the specificity of the antifungal mAb. Furthermore, the control conjugate containing the non-specific IgM mAb did not bind hyphae (data not shown). Interestingly, the anti AF mAb MPS 5.44 bound other pathogenic Aspergillus species such as Aspergillus niger, Aspergillus flavus and Aspergillus terreus with a similar high affinity, without wishing to be bound by theory or mechanism of action this might indicate that anti AF mAb MPS 5.44 interacts with a surface epitope shared by Aspergilli (FIG. 2D). In contrast, no binding was observed to either Candida albicans or Candida krusei.

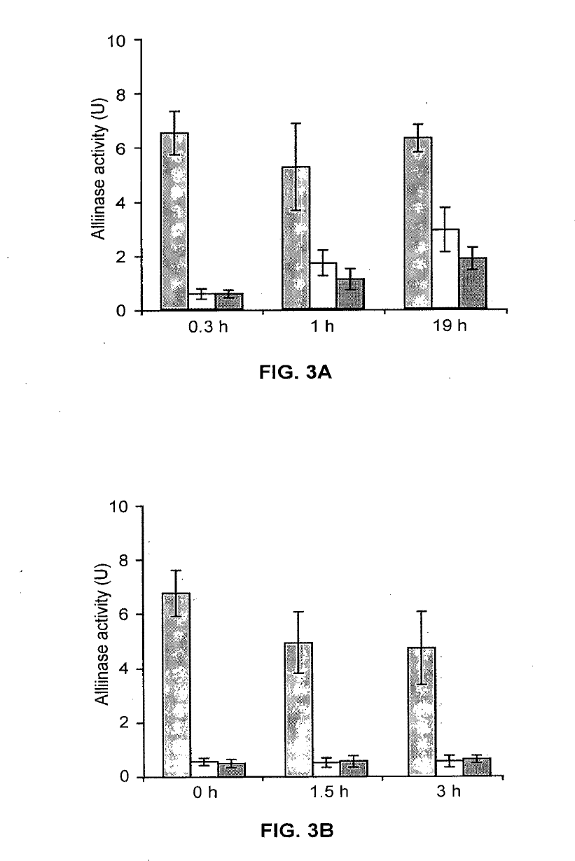

Specific binding of the conjugate to either AF conidia or hyphae was rapid and reached saturation in 20 mM. Further incubation for 1 hour or longer did not increase the binding of the conjugate (FIG. 3A). Importantly, the alliinase activity of the conjugates was preserved on the surface of conidia for at least 3 hours (FIG. 3B). Without wishing to be bound by theory of mechanism of action this might indicate that for at least 3 hours binding saturation is achieved, the conjugate does not undergo clearance from the fungal surface and the enzymatic activity of alliinase is retained at a similar level. The alliinase activity of the bound mAb-alliinase was significantly higher (p<0.001) than the alliinase activity of the non-specifically bound conjugates.

### Example 2

### Periodic Acid Oxidation of Aspergillus fumigatus Inhibits the Binding of the Antibody of the Invention to the Fungus

Immunoglobulins of class M (IgM) are frequently directed towards polysaccharides. In order to determine whether the antigen to which the antibody of the invention binds, comprises a polysaccharide, Aspergillus fumigatus hyphae were pretreated with periodate. Without wishing to be bound by theory or mechanism of action, oxidation by periodate modifies the polysaccharides of the fungi cell wall. Such a modification might therefore disturb the binding of a polysaccharide-specific mAbs.

Fresh conidia of AF293, 2×10⁶ ml were seeded in 96-well plates in RPMI/MOPS and were grown over night at 37° C. On the following day, 100 mM (21 mg/ml) of Na meta-periodate (NaIO₄) were added to the grown AF293 hyphae, and the plates were placed in the dark at 4° C. and incubated over night. Control plates were incubated with PBS. Alternatively, hyphae were fixed in both control and experimental plates with 3.7% formaldehyde/PBS for 30 min at RT, washed three times with PBS, and NaIO₄ was added as described above.

All Plates were then washed three times with PBS followed by incubation with 250 mM Glycine-buffer (pH 7-8 with HCl) for 1 h at RT, in the dark. Plates were then washed again with PBS (3 times) and then blocked with 1% hemoglobin (Hb) in TBST buffer for 1 h at 37° C.

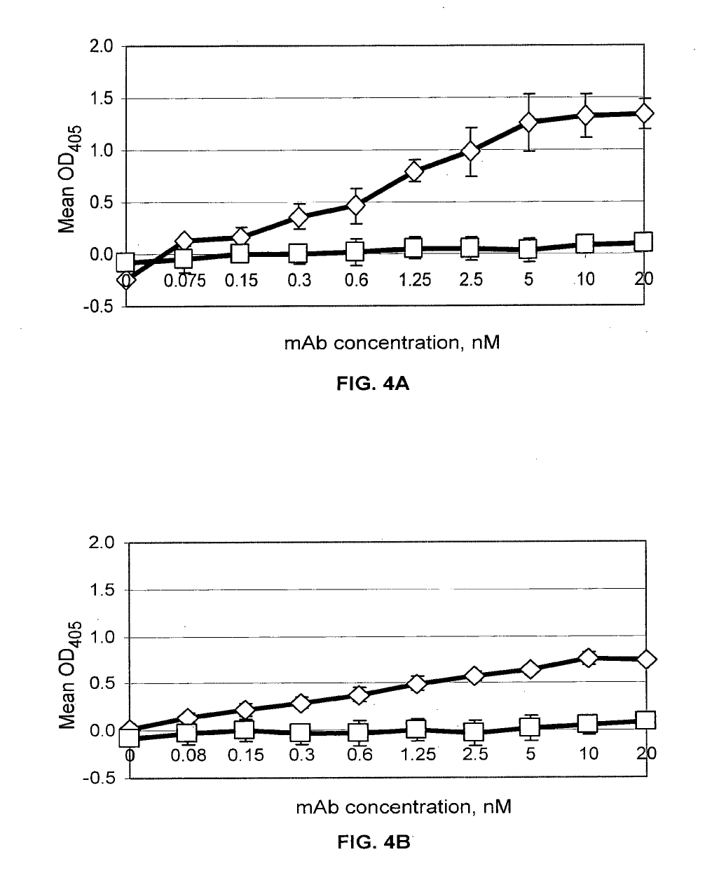

The binding of mAb to Aspergillus species was followed as described above in Example No. 1. It was found that the antibody of the invention binds Aspergillus fumigatus at concentrations as low as 1 nM, whereas periodic oxidation abrogates the binding completely (FIG. 4A).

Formaldehyde fixation of hyphae partially preserved the targeting sites for the antibody of the invention, however, periodic acid oxidation depleted the binding completely (FIG. 4B).

These results indicated that the antibody of the invention recognizes a unique carbohydrate structure on the surface of different Aspergillus species.

### Example 3

### In Vitro Fungicidal Properties of the MAB-Alliinase Conjugates

The antifungal activity of mAb (MPS5.44)-alliinase conjugates was determined according to the conditions of National Committee for Clinical Laboratory Standards (NCCLS) document M27-A. Resting conidia (3×10⁴ conidia/well) were seeded in 96-well plates and incubated for 4 hours at 37° C. with RPMI/MOPS (100 μl). mAb-alliinase conjugate was applied in serial 2-fold dilutions, in triplicates and incubated for 30 min at 37° C., then washed four times followed by the addition of alliin (0.5 mg/ml). Hyphal growth was monitored by microscopic observation as well as by optical density (OD₅₉₅), or by fluorescence of the strain CBS/DsRed (excitation at 540 nm, emission at 595 nm). The MIC of the conjugate was determined after 72 hours. The wells in which no fungal germination was detected, were scraped and plated on Sabouraud plates and the number of colonies was counted after 72 hours to determine the MFC. As a control, the antifungal activity was also determined by incubating AF as above with non-conjugated alliinase or with conjugates consisting of the non-specific mAb and alliinase.

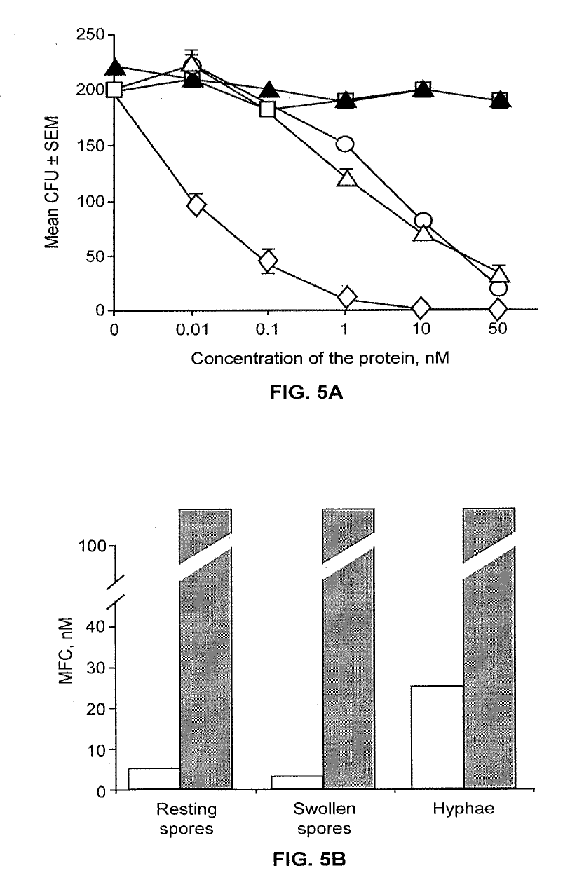

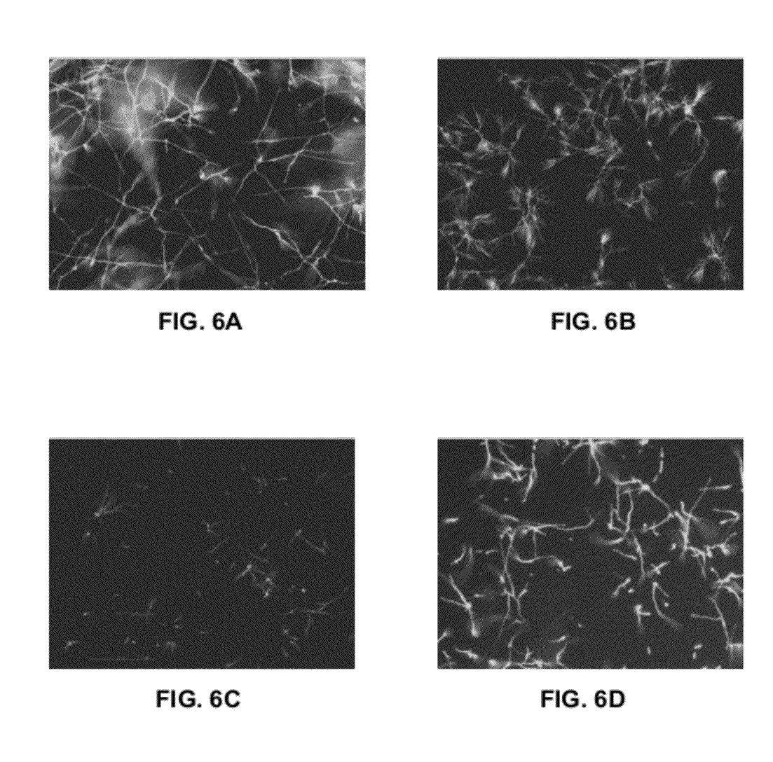

Results: The antifungal activity of the conjugate was determined on swollen conidia preincubated (30 min, 37° C.) with serial dilutions of the mAb-alliinase conjugate followed by washing of the unbound conjugate and addition of alliin. Complete sterility (minimal fungicidal concentration (MFC)) was achieved with conjugate concentrations as low as 5-10 nM. Curve fit analysis indicates statistically significant (p<0.01) differences between CFU (colony forming unit) counts determined after treatment with mAb-alliinase conjugate and alliin in comparison to treatments with the non-specific conjugate or with free alliinase and alliin (FIG. 5A). The minimal fungicidal concentration (MFC) for resting conidia treated likewise was similar, whereas for hyphae MFC was about 25 nM (FIG. 5B). This higher value may reflect a difference in fungal mass. In comparison, the MFC of pure allicin was 7.5 μg/ml for swollen conidia, 15 μg/ml for resting conidia and 30 μg/ml for hyphae (data not shown). Swollen conidia preincubated with unconjugated alliinase or with the non-specific mAb-alliinase conjugate followed by addition of alliin were not killed even at the highest examined concentration (50 nmol) (FIG. 5 A, B). Notably, neither mAb MPS5.44 alone nor conjugate without alliin inhibited germination of conidia or hyphae formation when applied for short periods (30 min followed by washing) (FIG. 6A). Prolonged incubations (19 h) of conidia with unconjugated mAb MPS5.44 (20 nmol), resulted in partial inhibition of hyphal formation (FIG. 6B). Conjugate applied for the same period of time but without alliin also had some inhibitory effect (FIG. 6C). Upon addition of alliin (0.5 mg/ml), the concentrations of the conjugate could be decreased to as low as 10 pmol for a similar suppression of fungal growth (FIG. 6D). In conclusion, the antibody alone or conjugate without alliin had low anti-AF activity. In the presence of alliin, the effective fungicidal concentrations of the conjugate were three orders of magnitude lower.

### Example 4

### Pulmonary Challenge and Determination of Fungal Infection in Mice

Eight week-old ICR female mice of body weight 25-28 grams, were maintained under specific pathogen-free conditions and handled under protocols approved by the Weizmann Institute Animal care Committee according to International guidelines. Mice were immunosuppressed and challenged as previously described by Vallon-Eberhard et al., 2008, Antimicrob. Agents Chemother. 52:3118-26. Briefly, cortisone (25 mg in 200 μl PBS) was injected i.p. on days 0 and 3. Before infection, mice were anesthetized with isoflurane, and 10⁷ DsRed conidia in 50 μl PBS were inoculated intra-nasally on day 0.

In a preliminary experiment, the optimal volumes and dosages of the conjugates and alliin that could be introduced by intratracheal instillation (i.t.) without causing animal discomfort, where determined. It was found that i.t. administrations of 50 nmol conjugate in 50 μl PBS followed by, 30 minutes later, 750 μg alliin in 25 μl PBS were well tolerated by the mice. A short term study of tissue burden was then performed. For this experiment two groups of mice (n=5 each) were infected as above. One hour after infection, the animals in the control group were mock treated by intratracheal instillation with PBS (50 μl). The second group was i.t administered with a solution of mAb-alliinase conjugate (50 nmol conjugate in 50 μl PBS). Thirty minutes later, alliin (750 μl in 25 μl PBS) was administered i.t. to both groups. All the infected animals survived the intratracheal administrations. On the fourth day of the experiment, the animals were euthanized and an infection read-out was carried out on fresh sections of inflated lungs using a confocal microscope. Fungal burden was determined in aliquots (20 and 100 μl) of the lung tissue homogenate (total volume, 2 ml PBS) by CFU enumeration on Sabouraud dextrose agar plates and extrapolated to the whole lung.

Results:

In a previous work (Vallon-Eberhard et al., 2008, Antimicrob. Agents Chemother. 52:3118-26) the intranasal route of pulmonary infection in immuno-suppressed mice as well as the optimal number of DsRed conidia required to obtain a reproducible rate of infection was established. In order to confirm the short term development of pulmonary fungal infection in the present study, two groups of mice (5 per group) were treated as described in Materials and Methods. The mice were euthanized on day 4 and the five lobes of their lungs were sliced and examined by confocal microscope to count the number of fluorescent fungal colonies. Animals in the control group that were treated with PBS and alliin but did not receive conjugate had large numbers (290±56) of fungal colonies throughout their lungs. One representative colony is shown on FIG. 6A. In comparison, mice treated with the conjugate and alliin had very few visible small colonies, (28±9.4) in their lungs, significantly lower (p<0.01) than the control group (FIG. 6B). Fungal burden was also determined in homogenates of lung lobes, revealing a high number of CFU's per lung (442±78) in the PBS treated group versus 3.3±3.3 CFU's in the conjugate treated group (p<0.01) which correlated well with the above microscopic enumeration. In conclusion, on day 4 post-challenge, the placebo treated mice had numerous hyphal colonies spread throughout their lungs whereas animals that received a single i.t. treatment of conjugate and alliin had almost no AF. Conjugate/alliin treatment significantly reduces lung fungal load in AF-infected mice.

### Example 5

### Pulmonary Challenge and Determination of Fungal Infection in Mice

### Animal Survival Experiments.

Animals infected as above were divided into groups (n=15):

G-1: control group (placebo) were administered i.t. with PBS (50 μl) one hour after infection, followed by (30 minutes later) the i.t. administration of PBS (25 μl);
G-2: control group (placebo) were administered i.t. with PBS (50 μl) one hour after infection, followed by (30 minutes later) the i.t. administration of alliin (750 μg in 25 μl PBS).
G-3: were administered i.t. with unconjugated mAb MPS 5.44 (50 nmol/50 μl) one hour after infection, followed by (30 minutes later) the i.t. administration of PBS (250);
G-4: were administered i.t. with mAb-alliinase conjugate (50 nmol in 50 μl PBS) one hour after infection, followed by (30 minutes later) the i.t. administration of PBS (25 μl);

G-5: were administered i.t. with mAb-alliinase conjugate (50 nmol in 50 μl PBS) one hour after infection, followed by (30 minutes later) the i.t. administration of alliin (750 μg/25 μl PBS);

G-6: were administered i.t. with unconjugated alliinase (50 nmol/50 μl) one hour after infection, followed by (30 minutes later) the i.t. administration of alliin (750 μg/25 μl PBS).
G-7: were administered i.t. with mAb-alliinase conjugate (50 nmol/50 μl PBS) 50 hours post-infection, followed by (30 min later) the i.t. administration of alliin (750 μg/25 μl PBS) This group was used in order to evaluate the treatment of fulminant invasive Aspergillus by the mAb-alliinase conjugate/alliin treatment of the invention.
G-8: (anti-fungal drug control; n=10) were administered i.t one hour after infection with Amphotericin B at a concentration of 1 mg/kg.

All treatments were repeated on days 4, 6 and 9 post-infection and animal survival in the different groups was followed for 36 days.

The evaluation of fungal infection in the lungs of mice that survived and were euthanized after 36 days or those which died during the survival experiment, was performed in two ways: one lobe was fixed in 4% PBS-buffered formalin, embedded in paraffin and cut into 5 μm thick sections. Sections were stained with H&E or periodic acid-Schiff (PAS) for fungal detection and examined microscopically. The remaining four lobes were homogenized in 2 ml PBS and aliquots were seeded on Sabouraud plates for CFU enumeration of fungal burden.

Statistical Analysis: Survival data were analyzed by the Kaplan-Meier method using GraphPad Prism 5 software (GraphPad Inc.). Differences in survival curves were assessed by the log-rank test. Data from CFU counts in the lungs were analyzed by unpaired two-tailed t-test with Welch's correction and by non-parametric two tailed Mann-Whitney U test._ELISA data were assessed for statistical significance by curve fit analysis.

Results:

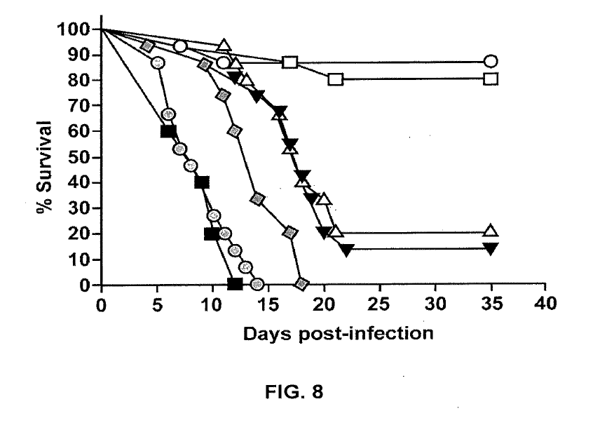

In order to assess the effect of treatment with conjugate and alliin on the long term survival (36 days) of AF-infected mice, an experiment was carried out with larger groups of animals (n=15) as described above. As shown in FIG. 8, 100% of the AF-infected mice in the control groups that received PBS instead of conjugate (G-1 and G-2) died by day 13 (MST=7 d). Infected mice treated with unconjugated mAbs (G-3), or conjugate without alliin (G-4) exhibited 80% mortality at 36 days (MST=18 d). Log rank testing indicated a statistically significant difference between the survival curves of placebo-treated animals versus those that were administered with either unconjugated mAb or conjugate without alliin (p<0.001). This may indicate that the mAb according to some embodiments of the invention is a protective antibody that can significantly prolong the survival of infected mice.

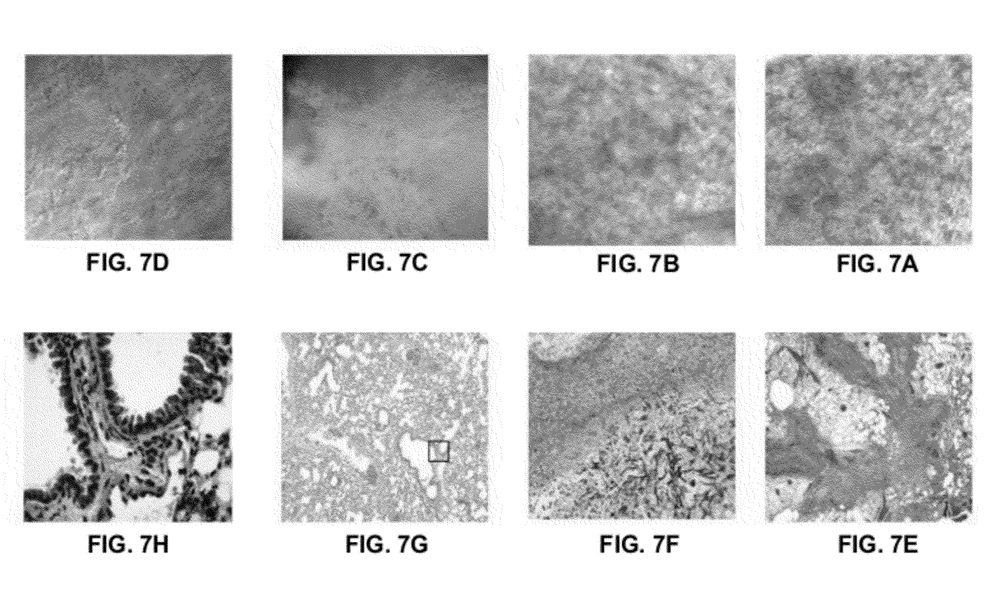

Importantly, the most impressive therapeutic effect (>85% survival) was seen with mice from G-5 that were treated with conjugate and alliin. Two of these mice died during early infection (days 7 and 11) but the rest survived for the duration of the experiment (36 d). To find out whether the efficacy of the conjugate-based treatment was antibody-dependent, animals in G-6 were treated with unconjugated alliinase and alliin (FIG. 8). The alliinase concentration (50 nmole in 50 μl) and its specific activity (120 U/nmol) were similar to that of the alliinase which was ligated in the conjugates. Following this treatment, G-6 mice began to die on day 4 post-infection and none survived for over 3 weeks (MST=14 d) (FIG. 8). The survival curves of the animals treated with non-conjugated alliinase and alliin (G-6) significantly differed from (i) the control treated mice (G-2) (p<0.01) as well as from (ii) the conjugate and alliin treated animals (G-5) (p<0.001). Without wishing to be bound by theory or mechanism of action, this result may imply that allicin produced in the bronchial space by the unconjugated alliinase had some antifungal activity, but was not as effective as that produced by the mAb-targeted alliinase anchored to the surface of the fungus (G-5). We also tested whether treatment with the conjugate and alliin could have a therapeutic effect in treating a more advanced pulmonary infection. For this purpose the intra-tracheal administrations of conjugate and alliin in the infected mice of G-7 we delayed and the treatment started 50 hours post-infection. Treatment was repeated on days 4, 6, and 9. As can be seen in FIG. 7, 80% of the mice in this group survived for the duration of the experiment (36 d), comparable to that of G-5 (p>0.05) in which the treatments with conjugate and alliin, as mentioned above, started on the day of infection. Thus, treatments with conjugate/alliin protected the majority of animals (>80%), even when started at a more advanced stage of infection. Interestingly, in both cases, treatment with conjugate/alliin was more effective as compared to the gold standard treatment with the antifungal drug AMB (group 8, MST=18 d) where only 30% of mice survived at the end of the experiment.

Lung tissues from sham (G-1, G-2) and conjugate (G-5, G-7) treated animals were examined by histology and fungal burden analysis for the presence of AF. The results indicate a high fungal burden (6700±2056 colonies per lung) and extensive invasive pulmonary aspergillosis in the lungs of placebo treated mice (FIGS. 7, C, E, F) in comparison to a very low fungal burden (4.3±2.3 colonies per lung, (p<0.01)) and no apparent fungal growth in the lungs of conjugate/alliin-treated mice euthanized at the end of the experiment (FIG. 7, D, G, H). No tissue damage was observed in either liver or kidneys of the conjugate/alliin treated mice that survived (data not shown).

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1. An isolated monoclonal antibody specific to Aspergillus, or an antibody fragment comprising at least an antigen-binding portion thereof, comprising the six complementarity determining region (CDR) sequences: CDR_{H1} (SEQ ID NO:1), CDR_{H2} (SEQ ID NO:2), CDR_{H3} (SEQ ID NO:14), CDR_{L1} (SEQ ID NO:3), CDR_{L2} (SEQ ID NO:4), and CDR_{L3} (SEQ ID NO:5).

2. The monoclonal antibody or fragment thereof according to claim 1 wherein the monoclonal antibody is produced by a hybridoma cell which has been deposited under Accession Number CNCM 1-4377.

3-6. (canceled)

7. The monoclonal antibody or fragment thereof according to claim 1 comprising a heavy chain variable region comprising the amino acid sequences of CDR_{H1} (SEQ ID NO:1), CDR_{H2} (SEQ ID NO:2) and CDR_{H3} (SEQ ID NO:14), and a Kappa light chain variable region comprising the amino acid sequences of CDR_{L1} (SEQ ID NO:3), CDR_{L2} (SEQ ID NO:4) and CDR_{L3} (SEQ ID NO:5).

8. The monoclonal antibody or fragment thereof according to claim 1 comprising a light chain variable region which comprises the amino acid sequence of SEQ ID NO:6 and a heavy chain variable region which comprises the amino acid sequence of SEQ ID NO:7.

9-14. (canceled)

15. The monoclonal antibody or fragment thereof, according to claim 1 which specifically binds at least one Aspergillus species selected from the group consisting of Aspergillus fumigatus, Aspergillus niger, Aspergillus flavus, Aspergillus terreus, Aspergillus caesiellus, Aspergillus candidus, Aspergillus carneus, Aspergillus chevalieri, Aspergillus clavatus, Aspergillus deflectus, Aspergillus flavipes, Aspergillus glaucus, Aspergillus granulosus, Aspergillus nidulans, Aspergillus ochraceus, Aspergillus oryzae, Aspergillus parasiticus, Aspergillus penicilloides, Aspergillus restrictus, Aspergillus sojae, Aspergillus sydowi, Aspergillus tamari, Aspergillus ustus, Aspergillus versicolor and Aspergillus wentii.

16. (canceled)

17. The monoclonal antibody or fragment thereof, according to claim 15 which specifically binds the Aspergillus species Aspergillus fumigatus, Aspergillus niger, Aspergillus flavus and Aspergillus terreus.

18. (canceled)

19. The antibody fragment according to claim 1 selected from the group consisting of: a single chain, an F(ab′)₂, an F(ab), and Fv.

20. The antibody fragment according to claim 1, wherein the monoclonal antibody is a humanized or a chimeric antibody.

21. (canceled)

22. A conjugate or a fusion protein comprising at least one antibody or fragment thereof, according to claim 1.

23. The conjugate or fusion protein according to claim 22, further comprising an enzymatically active form of alliinase.

24-25. (canceled)

26. An isolated polynucleotide sequence encoding a monoclonal antibody or fragment according to claim 1.

27-32. (canceled)

33. A vector comprising the polynucleotide sequence according to claim 26.

34. A host cell comprising the vector according to claim 33.

35. A hybridoma cell line deposited under Accession No. CNCM I-4377.

36. A pharmaceutical composition comprising at least one monoclonal antibody or an antibody fragment comprising at least an antigen-binding portion thereof, according to claim 1, and a pharmaceutically acceptable carrier.

37. A method of increasing the survival rate of a subject infected with an Aspergillus species comprising administering to a subject in need thereof a pharmaceutical composition according to claim 36.

38. A method of treating a disease or condition caused by Aspergillus species in a subject comprising administering to the subject a pharmaceutical composition according to claim 36.

39. The method according to claim 38 wherein the disease or condition is selected from the group consisting of invasive aspergillosis, allergic bronchopulmonary aspergillosis, chronic necrotizing aspergillus pneumonia and pulmonary aspergilloma.

40. The method according to claim 38 comprising administering the pharmaceutical composition at an advanced stage of Aspergillus infection.

41. The method according to claim 38 wherein the pharmaceutical composition comprises a conjugate or a fusion protein of the enzyme alliinase with the monoclonal antibody or the antigen binding fragment thereof, and the method further comprises administration of alliin.

42. (canceled)
